(19) United States
(12) Patent Application Publication
(10) Pub. No.: US 2024/0342083 A1
(43) Pub. Date: Oct. 17, 2024

(54) ANTI-PD-1 ANTIBODY PHARMACEUTICAL COMPOSITION AND USE THEREOF

(71) Applicant: SHANGHAI JUNSHI BIOSCIENCES CO., LTD., SHANGHAI (CN)

(72) Inventors: Hongchuan LIU, SUZHOU (CN); Peixiang LIU, SUZHOU (CN); Xiaojie DU, SUZHOU (CN); Qin MENG, SUZHOU (CN); Hui LIU, SUZHOU (CN); Hui FENG, SUZHOU (CN)

(73) Assignee: SHANGHAI JUNSHI BIOSCIENCES CO., LTD., SHANGHAI (CN)

(21) Appl. No.: 18/291,935

(22) PCT Filed: Jul. 29, 2022
(86) PCT No.: PCT/CN2022/108825

§ 371 Date: Jan. 25, 2024

(30) Foreign Application Priority Data
Jul. 29, 2021 (CN) 202110863978.0

(51) Int. Cl. A61K 9/08; A61K 9/00; A61K 39/00; A61K 47/02; A61K 47/12; A61K 47/18; A61K 47/26; A61P 35/00; C07K 16/28

(52) U.S. Cl. CPC A61K 9/08; A61K 9/0019; A61K 47/02; A61K 47/12; A61K 47/183; A61K 47/26; A61P 35/00; C07K 16/2818; A61K 2039/505

(57) ABSTRACT

The present disclosure provides a stable anti-PD-1 antibody pharmaceutical composition and use thereof. The pharmaceutical composition includes a buffer and an anti-PD-1 antibody or an antigen-binding fragment thereof; and the anti-PD-1 antibody or the antigen-binding fragment thereof has a concentration of about 100-250 mg/mL, and includes an LCDR1, an LCDR2 and an LCDR3 having amino acid sequences set forth in SEQ ID NO: 1, SEQ ID NO: 2 and SEQ ID NO: 3, respectively, and an HCDR1, an HCDR2 and an HCDR3 having amino acid sequences set forth in SEQ ID NO: 4, SEQ ID NO: 5 and SEQ ID NO: 6, respectively; and and the pharmaceutical composition has a pH of about 5.0-6.5. The present disclosure also provides an injection including the pharmaceutical composition and use of the pharmaceutical composition and the injection in preparing a medicament for treating a disease or disorder by eliminating, inhibiting or reducing PD-1 activity.

### FIELD

The present disclosure relates to the field of therapeutic pharmaceutical compositions, in particular to an anti-PD-1 antibody pharmaceutical composition and use thereof.

### BACKGROUND

Immune escape is one of the characteristics of cancer. Ahmadzadeh, M. et al. disclosed in Blood, 114: 1537-44 that tumor-specific T lymphocytes are often present in the tumor microenvironment, draining lymph nodes and peripheral blood, but are generally unable to control tumor progression due to the network of immunosuppressive mechanisms present in the tumor microenvironment. CD8⁺ tumor infiltrating T lymphocytes (TILs) generally express activation-induced inhibitory receptors, including CTLA-4 and PD-1, while tumor cells often express immunosuppressive ligands, including PD-1 ligand 1 (PD-L1, also called B7-H1 or CD274), which inhibits activation and effector functions of T cells. In the inhibitory mechanism, PD-1 and its ligands have become an important pathway for tumor cells to suppress activated T cells in the tumor microenvironment.

Programmed death receptor 1 (PD-1) plays an important role in immune regulation and maintenance of peripheral tolerance. PD-1 is expressed primarily in activated T and B cells and functions to inhibit the activation of lymphocytes, which is a normal peripheral tissue tolerance mechanism of the immune system that prevents over-reactive immunity. However, the activated T cells infiltrated in the tumor microenvironment highly express PD-1 molecules, and inflammatory factors secreted by the activated leukocytes can induce the tumor cells to highly express ligands PD-L1 and PD-L2 of PD-1, resulting in the continuous activation of the PD-1 pathway of the activated T cells in the tumor microenvironment, and the suppression of T cell function to kill tumor cells. Therapeutic PD-1 antibodies can block this pathway, partially restore the function of T cells, and enable the activated T cells to continuously kill tumor cells.

Blocking the PD-1/PD-L1 pathway has proven to be an effective way to induce a durable anti-tumor response in various cancer indications over the last decade. Monoclonal antibodies (mAbs) blocking the PD/PD-L1 pathway can enhance activation and effector functions of tumor specific T cells, reduce tumor burden, and improve survival rate.

Antibody pharmaceutical formulations should be stable for a long period of time, and contain a safe and effective amount of the pharmaceutical formulation. Due to the specific structure and properties of the antibody, antibody drugs need an environment that enables their stability during the preparation, storage and transportation processes. For different kinds of proteins and different kinds of antibodies, their physicochemical properties, degradation reactions and the like are different. Therefore, the formulas of buffers, excipients and the like of the antibody pharmaceutical formulations are also different.

Subcutaneous (SC) injection is a preferred embodiment to improve compliance and convenience of administration for tumor patients, but its effectiveness requires a relatively high dose, therefore, there is a need to prepare high-concentration formulations. However, the preparation of high-concentration antibody formulations is often accompanied by many difficulties. For example, such formulations have high viscosity, which makes it difficult to draw and push the drug with a syringe, and results in the high residue of the drug in the container and syringe, to cause a large deviation of the administered dose, pain at the injection site, and the like. Furthermore, the high viscosity of the formulations also causes serious process problems during its production, such as the need for extremely high pressures in the concentration and filtration stages, or the inability to pass through the filtration membrane. For another example, the high-concentration antibody proteins in the formulation are easy to aggregate, which causes instability of the formulation, facilitates the formation of insoluble particles, increases drug immunogenicity, increases drug side effects, and the like.

Therefore, there is still a need in the art to develop a high-concentration antibody formulation targeting human programmed death receptor 1 to meet the manufacturing and clinical application requirements for the high-concentration antibody having long-term stability, no aggregation, low viscosity and the like.

### SUMMARY

The pharmaceutical composition described herein is a highly stable pharmaceutical composition including an antibody specifically binding to PD-1. In particular, the present disclosure develops a high-concentration antibody formulation by selecting a suitable buffer system and pH, optimizing stabilizer and surfactant, and carrying out pharmacokinetics and pharmacodynamics studies, which can be used for subcutaneous administration, and has long-term stability, no aggregation and ultra-low viscosity.

The present disclosure provides a pharmaceutical composition including: (1) a buffer; and (2) an anti-PD-1 antibody or an antigen-binding fragment thereof.

In some embodiments, the anti-PD-1 antibody or the antigen-binding fragment thereof includes an LCDR1, an LCDR2 and an LCDR3 having amino acid sequences set forth in SEQ ID NO: 1, SEQ ID NO: 2 and SEQ ID NO: 3, respectively, and an HCDR1, an HCDR2 and an HCDR3 having amino acid sequences set forth in SEQ ID NO: 4, SEQ ID NO: 5 and SEQ ID NO: 6, respectively.

In some embodiments, the anti-PD-1 antibody or the antigen-binding fragment thereof is selected from a murine antibody or an antigen-binding fragment thereof, a chimeric antibody or an antigen-binding fragment thereof, and a humanized antibody or an antigen-binding fragment thereof, preferably a humanized antibody or an antigen-binding fragment thereof.

In some embodiments, the anti-PD-1 antibody or the antigen-binding fragment thereof includes a light chain variable region set forth in SEQ ID NO: 7 and a heavy chain variable region set forth in SEQ ID NO: 8.

In some embodiments, the anti-PD-1 antibody includes a light chain amino acid sequence set forth in SEQ ID NO: 9 and a heavy chain amino acid sequence set forth in SEQ ID NO: 10.

In some embodiments, the anti-PD-1 antibody or the antigen-binding fragment thereof in the pharmaceutical composition has a concentration of about 100-250 mg/mL, preferably about 150-250 mg/mL, and more preferably about 150-200 mg/mL; more preferably, the anti-PD-1 antibody or the antigen-binding fragment thereof has a concentration of about 100 mg/mL, 110 mg/mL, 120 mg/mL, 130 mg/mL, 140 mg/mL, 150 mg/mL, 160 mg/mL, 170 mg/mL, 175 mg/mL, 180 mg/mL, 185 mg/mL, 190 mg/mL, 195 mg/mL, 200 mg/mL, 210 mg/mL or 220 mg/mL, preferably about 180 mg/mL, 185 mg/mL, 190 mg/mL or 195 mg/mL.

In some embodiments, the pharmaceutical composition has a pH of about 5.0-6.5, preferably about 5.5-6.2, more preferably about 5.9-6.1, and even more preferably about 6.0.

In some embodiments, the pharmaceutical composition has an osmotic pressure in a range of 260-320 mOsm/kg, preferably in a range of 290-310 mOsm/kg.

In some embodiments, the pharmaceutical composition has a viscosity of ≤8.0 cP as measured at about 25° C.

In some embodiments, the buffer is selected from one or more of an acetic acid buffer, a citric acid buffer and a histidine buffer, preferably a histidine buffer.

In some embodiments, the histidine buffer is selected from a histidine-histidine hydrochloride buffer and a histidine-histidine acetate buffer, preferably a histidine-histidine hydrochloride buffer.

In some embodiments, the histidine buffer is a histidine-histidine hydrochloride buffer. In some embodiments, the histidine-histidine hydrochloride buffer is prepared by histidine and histidine hydrochloride, preferably L-histidine and L-histidine monohydrochloride. In some embodiments, the histidine buffer is prepared by 1-30 mM L-histidine and 1-30 mM L-histidine monohydrochloride. In some embodiments, the histidine buffer is prepared by histidine and histidine hydrochloride in a molar ratio of 1:1-1:4. In some embodiments, the histidine buffer is prepared by histidine and histidine hydrochloride in a molar ratio of about 1:1. In some embodiments, the histidine buffer is prepared by histidine and histidine hydrochloride in a molar ratio of about 1:3. In some embodiments, the histidine formulation is a histidine buffer having a pH of about 5.5 prepared by about 4.5 mM L-histidine and about 15.5 mM L-histidine monohydrochloride. In some embodiments, the histidine formulation is a histidine buffer having a pH of about 5.5 prepared by about 7.5 mM L-histidine and about 22.5 mM L-histidine monohydrochloride. In some embodiments, the histidine formulation is a histidine buffer having a pH of about 6.0 prepared by about 10 mM histidine and about 10 mM histidine hydrochloride.

In some embodiments, the histidine buffer is a histidine-histidine acetate buffer, preferably, histidine and histidine acetate are in a molar ratio of 1:1 to 1.5:1; preferably, such a buffer has a pH of 6.0±0.3, preferably about 6.0; and preferably, such a buffer contains 10-15 mM histidine and 10-15 mM histidine acetate.

In some embodiments, the buffer is an acetic acid buffer; preferably, the acetic acid buffer is an acetic acid-sodium acetate buffer or an acetic acid-potassium acetate buffer, preferably an acetic acid-sodium acetate buffer. In some embodiments, the acetic acid buffer is prepared by 1-30 mM acetic acid and 1-30 mM sodium acetate. In some embodiments, the acetic acid buffer is prepared by acetic acid and sodium acetate in a molar ratio of about 1:2.1. In some embodiments, the acetic acid buffer is prepared by acetic acid and sodium acetate in a molar ratio of about 1:5.7. In some embodiments, the acetic acid buffer is an acetic acid buffer having a pH of about 5.0 prepared by about 6.5 mM acetic acid and about 13.5 mM sodium acetate. In some embodiments, the acetic acid buffer is an acetic acid buffer having a pH of about 5.5 prepared by about 3 mM acetic acid and about 17 mM sodium acetate.

In some embodiments, the buffer is a citric acid buffer, preferably a citric acid-sodium citrate buffer. In some embodiments, the citric acid buffer is prepared by 1-30 mM citric acid and 1-30 mM sodium citrate. In some embodiments, the citric acid buffer is prepared by citric acid and sodium citrate in a molar ratio of about 1:1 to 1:4. In some embodiments, the citric acid buffer is a citric acid buffer having a pH of about 6.0 prepared by about 5.0 mM citric acid and about 15.0 mM sodium citrate. In some embodiments, the citric acid buffer is a citric acid buffer having a pH of about 6.0 prepared by about 10 mM citric acid and about 10 mM sodium citrate.

In some embodiments, the buffer has a concentration of about 5-100 mM, preferably about 10-50 mM, preferably about 10-30 mM, and preferably about 15-25 mM; and a non-limiting example of the concentration of the buffer is about 10 mM, 15 mM, 20 mM, 25 mM, 30 mM, 40 mM, 45 mM or 50 mM or in a range with any two of the values as endpoints, preferably about 15 mM, 20 mM or 25 mM.

In some embodiments, the buffer has a pH of about 5.0-6.5, preferably about 5.5-6.5, preferably about 5.5-6.2, and more preferably about 5.9-6.1; and a non-limiting example of the pH of the buffer is about 5.0, 5.1, 5.2, 5.3, 5.4, 5.5, 5.6, 5.7, 5.8, 5.9, 6.0, 6.1, 6.2, 6.3, 6.4 or 6.5, preferably about 5.9, 6.0 or 6.1.

In some embodiments, the pharmaceutical composition further includes a stabilizer selected from one or more of arginine, an arginine salt, sodium chloride, mannitol, sorbitol, sucrose, glycine and trehalose; preferably, the arginine salt is arginine hydrochloride.

In some embodiments, the stabilizer has a concentration of about 10-400 mM, preferably about 100-250 mM, preferably about 120-220 mM, and preferably about 130-180 mM; and a non-limiting example of the concentration of the stabilizer is about 100 mM, 110 mM, 120 mM, 130 mM, 140 mM, 145 mM, 150 mM, 160 mM, 170 mM, 180 mM, 190 mM, 200 mM, 210 mM, 220 mM or 230 mM or in a range with any two of the values as endpoints, preferably about 140 mM, 150 mM or 160 mM.

In some embodiments, the stabilizer is arginine or an arginine salt having a concentration of about 120-220 mM; or the stabilizer is a combination of arginine hydrochloride having a concentration of about 30-100 mM and sucrose having a concentration of about 100-180 mM; or the stabilizer is a combination of arginine hydrochloride having a concentration of about 30-100 mM and glycine having a concentration of about 50-150 mM; preferably, the stabilizer is arginine or an arginine salt having a concentration of about 130-180 mM; or the stabilizer is a combination of arginine hydrochloride having a concentration of about 30-70 mM and sucrose having a concentration of about 110-170 mM; or the stabilizer is a combination of arginine hydrochloride having a concentration of about 30-70 mM and glycine having a concentration of about 80-120 mM; preferably, the arginine salt is arginine hydrochloride.

In some embodiments, the stabilizer is arginine or an arginine salt. In some embodiments, the stabilizer is arginine or an arginine salt having a concentration of about 30-250 mM, preferably about 100-250 mM, preferably about 120-220 mM, preferably about 130-180 MM, and preferably about 140-160 mM; and a non-limiting example of the concentration of the arginine or the arginine salt is about 100 mM, 110 mM, 120 mM, 125 mM, 130 mM, 135 mM, 140 mM, 145 mM, 150 mM, 155 mM, 160 mM, 170 mM, 180 mM, 190 mM or 200 mM, preferably about 135 mM, 140 mM, 145 mM, 150 mM or 155 mM; preferably, the arginine salt is arginine hydrochloride.

In some embodiments, the stabilizer is sucrose. In some embodiments, the stabilizer is sucrose having a concentration of about 100-300 mM, preferably about 150-300 mM, and preferably about 200-280 mM; and a non-limiting example of the concentration of the sucrose is about 200 mM, 210 mM, 220 mM, 230 mM, 240 mM, 250 mM, 260 mM, 270 mM or 280 mM, preferably about 220 mM.

In some embodiments, the stabilizer is trehalose. In some embodiments, the stabilizer is trehalose having a concentration of about 100-300 mM, preferably about 150-300 mM, and preferably about 200-280 mM; and a non-limiting example of the concentration of the trehalose is about 180 mM, 200 mM, 210 mM, 220 mM, 230 mM, 240 mM, 250 mM, 260 mM, 270 mM or 280 mM, preferably about 220 mM.

In some embodiments, the stabilizer is sodium chloride. In some embodiments, the stabilizer is sodium chloride having a concentration of about 30-200 mM, preferably about 50-190 mM, preferably about 100-180 mM, preferably about 120-170 mM, and preferably about 130-150 mM; and a non-limiting example of the concentration of the sodium chloride is about 100 mM, 110 mM, 120 mM, 125 mM, 130 mM, 135 mM, 140 mM, 145 mM, 150 mM, 155 mM, 160 mM, 170 mM, 180 mM, 190 mM or 200 mM, preferably about 135 mM or 140 MM.

In some embodiments, the stabilizer is mannitol. In some embodiments, the stabilizer is mannitol having a concentration of about 100-300 mM, preferably about 150-300 mM, and preferably about 200-280 mM; and a non-limiting example of the concentration of the mannitol is about 200 mM, 210 mM, 220 mM, 230 mM, 240 mM, 250 mM, 260 mM, 270 mM or 280 mM, preferably about 240 mM.

In some embodiments, the stabilizer is sorbitol. In some embodiments, the stabilizer is sorbitol having a concentration of about 100-300 mM, preferably about 150-300 mM, and preferably about 200-280 mM; and a non-limiting example of the concentration of the sorbitol is about 200 mM, 210 mM, 220 mM, 230 mM, 240 mM, 250 mM, 260 mM, 270 mM or 280 mM, preferably about 240 mM.

In some embodiments, the stabilizer is a combination of sodium chloride and mannitol. In some embodiments, the stabilizer is a combination of about 30-200 mM sodium chloride and about 30-200 mM mannitol, preferably a combination of about 30-100 mM sodium chloride and about 100-180 mM mannitol, and preferably a combination of about 30-70 mM sodium chloride and about 120-180 mM mannitol; and a non-limiting example of the stabilizer is a combination of about 50 mM sodium chloride and about 140 mM mannitol, or a combination of about 50 mM sodium chloride and about 150 mM mannitol.

In some embodiments, the stabilizer is a combination of arginine hydrochloride and sucrose. In some embodiments, the stabilizer is a combination of about 30-200 mM arginine hydrochloride and about 30-200 mM sucrose, preferably a combination of about 30-100 mM arginine hydrochloride and about 100-180 mM sucrose, and preferably a combination of about 30-70 mM arginine hydrochloride and about 110-170 mM sucrose; a non-limiting example of the stabilizer is a combination of about 50 mM arginine hydrochloride and about 130 mM sucrose; and a non-limiting example of the stabilizer is a combination of about 50 mM arginine hydrochloride and about 140 mM sucrose, or a combination of about 50 mM arginine hydrochloride and about 150 mM sucrose.

In some embodiments, the stabilizer is a combination of arginine hydrochloride and glycine. In some embodiments, the stabilizer is a combination of about 30-200 mM arginine hydrochloride and about 30-200 mM glycine, preferably a combination of about 30-100 mM arginine hydrochloride and about 50-150 mM glycine, and preferably a combination of about 30-70 mM arginine hydrochloride and about 80-120 mM glycine; and a non-limiting example of the stabilizer is a combination of about 50 mM arginine hydrochloride and about 100 mM glycine, or a combination of about 50 mM arginine hydrochloride and about 110 mM glycine.

In some embodiments, the stabilizer is a combination of sodium chloride and sucrose. In some embodiments, the stabilizer is a combination of about 30-200 mM sodium chloride and about 30-200 mM sucrose, preferably a combination of about 30-100 mM sodium chloride and about 100-180 mM sucrose, and preferably a combination of about 30-70 mM sodium chloride and about 100-150 mM sucrose; and a non-limiting example of the stabilizer is a combination of about 50 mM sodium chloride and about 120 mM sucrose, or a combination of about 50 mM sodium chloride and about 130 mM sucrose.

In some embodiments, the stabilizer is a combination of sodium chloride and trehalose. In some embodiments, the stabilizer is a combination of about 30-200 mM sodium chloride and about 30-200 mM trehalose, preferably a combination of about 40-150 mM sodium chloride and about 40-180 mM trehalose, and preferably a combination of about 40-100 mM sodium chloride and about 80-160 mM trehalose; a non-limiting example of the stabilizer is a combination of about 50 mM sodium chloride and about 120 mM trehalose, or a combination of about 50 mM sodium chloride and about 140 mM trehalose.

In some embodiments, the pharmaceutical composition further includes a surfactant selected from one or more of polysorbate 80, polysorbate 20 and poloxamer 188.

In some embodiments, the surfactant is selected from polysorbate 80.

In some embodiments, the surfactant is selected from polysorbate 20.

In some embodiments, based on w/v, the surfactant has a concentration of about 0.001%-0.1%, preferably about 0.01%-0.1%, preferably about 0.02%-0.08%, and more preferably about 0.02%-0.06%; and as a non-limiting example, the surfactant has a concentration of about 0.02%, 0.04% or 0.08%, preferably about 0.04%.

In some embodiments, the pharmaceutical composition includes or consists of components as shown in any one of (1)-(8):
(1) (a) about 150-250 mg/mL of the anti-PD-1 antibody or the antigen-binding fragment thereof; (b) about 10-30 mM histidine buffer having a pH of about 5.5-6.5; (c) about 120-220 mM arginine or arginine salt; and (d) about 0.01%-0.1% (w/v) polysorbate 80; or (2) (a) about 150-250 mg/mL of the anti-PD-1 antibody or the antigen-binding fragment thereof; (b) about 10-30 mM histidine buffer having a pH of about 5.5-6.5; (c) a stabilizer, being a combination of arginine hydrochloride having a concentration of about 30-100 mM and sucrose having a concentration of about 100-180 mM; and (d) about 0.01%-0.1% (w/v) polysorbate 80; or
(3) (a) about 150-250 mg/mL of the anti-PD-1 antibody or the antigen-binding fragment thereof; (b) about 10-30 mM histidine buffer having a pH of about 5.5-6.5; (c) a stabilizer, being a combination of arginine hydrochloride having a concentration of about 30-100 mM and glycine having a concentration of about 50-150 mM; and (d) about 0.01%-0.1% (w/v) polysorbate 80; or
(4) (a) about 150-200 mg/mL of the anti-PD-1 antibody or the antigen-binding fragment thereof; (b) about 10-30 mM acetic acid buffer having a pH of about 5.5-6.0; (c) about 130-180 mM arginine or arginine hydrochloride; and (d) about 0.02%-0.08% (w/v) polysorbate 80; or (5) (a) about 150-200 mg/mL of the anti-PD-1 antibody or the antigen-binding fragment thereof; (b) about 10-30 mM acetic acid buffer having a pH of about 5.5-6.0; (c) a stabilizer, being a combination of arginine hydrochloride having a concentration of about 30-70 mM and sucrose having a concentration of about 110-170 mM; and (d) about 0.02%-0.08% (w/v) polysorbate 80; or
(6) (a) about 150-200 mg/mL of the anti-PD-1 antibody or the antigen-binding fragment thereof; (b) about 10-30 mM acetic acid buffer having a pH of about 5.5-6.0; (c) a stabilizer, being a combination of arginine hydrochloride having a concentration of about 30-70 mM and glycine having a concentration of about 80-120 mM; and (d) about 0.02%-0.08% (w/v) polysorbate 80;
(7) (a) about 180 mg/mL of an anti-PD-1 antibody including a light chain amino acid sequence set forth in SEQ ID NO: 9 and a heavy chain amino acid sequence set forth in SEQ ID NO: 10; (b) about 20 mM histidine buffer having a pH of about 6.0; (c) about 140 mM arginine hydrochloride; and (d) about 0.02% (w/v) polysorbate 80; or
(8) (a) about 180 mg/mL of an anti-PD-1 antibody including a light chain amino acid sequence set forth in SEQ ID NO: 9 and a heavy chain amino acid sequence set forth in SEQ ID NO: 10; (b) about 20 mM histidine buffer having a pH of about 6.0; (c) about 150 mM arginine hydrochloride; and (d) about 0.04% (w/v) polysorbate 80.

In some embodiments, the present disclosure provides a pharmaceutical composition including a buffer, an anti-PD-1 antibody or an antigen-binding fragment thereof, a stabilizer, and a surfactant; and the anti-PD-1 antibody includes a light chain amino acid sequence set forth in SEQ ID NO: 9 and a heavy chain amino acid sequence set forth in SEQ ID NO: 10; the anti-PD-1 antibody or the antigen binding fragment thereof has a concentration of 150-200 mg/mL; and the pharmaceutical composition has a pH of 5.9-6.1 and an osmotic pressure in a range of 260-320 mOsm/kg, preferably 290-310 mOsm/kg. Preferably, in the pharmaceutical composition, the buffer is a histidine buffer having a concentration of 15-25 mM and a pH of about 5.9-6.1, preferably about 6.0. Preferably, in the pharmaceutical composition, the stabilizer is arginine hydrochloride having a concentration of about 140-160 mM, preferably about 150 mM. Preferably, in the pharmaceutical composition, the surfactant is polysorbate 80 having a concentration of preferably 0.02%-0.06% (w/v), preferably about 0.04% (w/v). Preferably, the pharmaceutical composition has a viscosity of ≤8.0 cP, more preferably ≤7.0 cP, as measured at about 25° C.

In some embodiments, the pharmaceutical composition described in any one of the embodiments herein is a liquid formulation or a lyophilized formulation.

In some embodiments, the pharmaceutical composition is a liquid formulation.

In some embodiments, the liquid formulation or the lyophilized formulation is stably stored at 2-8° C. for at least 3 months, at least 6 months, at least 12 months, at least 18 months or at least 24 months.

In some embodiments, the liquid formulation or the lyophilized formulation is stably stored at 40° C. for at least 7 days, at least 14 days or at least 28 days.

The present disclosure further provides an injection including the pharmaceutical composition described in any one of the embodiments herein and a sodium chloride solution or a glucose solution; and preferably, the sodium chloride solution has a concentration of about 0.85%-0.9% (w/v); preferably, the glucose solution has a concentration of about 5%-25% (w/v), more preferably about 5%-10% (w/v); preferably, in the injection, the anti-PD-1 antibody has a concentration of about 0.5-50 mg/mL, more preferably about 0.5-20 mg/mL; and the injection has a pH of about 5.0-6.5, preferably about 5.5-6.2.

In some embodiments, the pharmaceutical composition or the injection is administered by subcutaneous injection.

The present disclosure further provides use of the pharmaceutical composition or the injection described in any one of the embodiments herein in preparing a drug for treating a disease or a disorder by eliminating, inhibiting or reducing PD-1 activity.

The present disclosure further provides the pharmaceutical composition or the injection described in any one of the embodiments herein for treating a disease or a disorder by eliminating, inhibiting or reducing PD-1 activity.

The present disclosure further provides a method for treating a disease or a disorder by eliminating, inhibiting or reducing PD-1 activity, which includes administering to a subject in need thereof the pharmaceutical composition or the injection described in any one of the embodiments herein.

In some embodiments, the disease or the disorder is selected from cancer, infectious diseases and inflammatory diseases.

The present disclosure further provides a method for reducing the viscosity of a high-concentration antibody pharmaceutical formulation, and the antibody in the antibody pharmaceutical formulation has a concentration of ≥150 mg/mL, such as in a range of 150-250 mg/mL, and the method includes preparing the high-concentration antibody pharmaceutical formulation using arginine hydrochloride, sodium chloride, or sucrose and arginine hydrochloride as a stabilizer and using a histidine buffer as a buffer. Preferably, arginine hydrochloride is used in an amount and it has a concentration of about 100-200 mM, preferably about 140-160 mM in the prepared antibody pharmaceutical formulation. Preferably, sodium chloride is used in an amount and it has a concentration of about 100-200 mM, preferably about 140-160 mM in the prepared antibody pharmaceutical formulation. Preferably, when a mixture of sucrose and arginine hydrochloride is used as a stabilizer, sucrose is used in an amount and it has a concentration of about 100-180 mM, preferably about 110-150 mM in the prepared antibody pharmaceutical formulation; and arginine hydrochloride is used in an amount and it has a concentration of about 30-80 mM, preferably about 30-60 mM in the prepared antibody pharmaceutical formulation. Preferably, the histidine buffer used has a pH of 5.0-6.5, preferably 5.5-6.2, and more preferably 5.9-6.1. Preferably, the histidine buffer used is used in an amount and it has a concentration of 15-25 mM, preferably about 20 mM in the prepared antibody pharmaceutical formulation. Preferably, the antibody is the anti-PD-1 antibody described in any one of the embodiments herein. Preferably, the method for reducing the viscosity of the high-concentration antibody pharmaceutical formulation can reduce the viscosity of the prepared antibody pharmaceutical formulation to below about 8.0 cP (as measured at about 25° C.). In some embodiments, the method further includes adding the surfactant described in any one of the embodiments herein, preferably 0.02%-0.06% (w/v) polysorbate 80, to the antibody pharmaceutical formulation.

In some embodiments, the present disclosure further provides use of arginine hydrochloride, sodium chloride or sucrose with arginine hydrochloride as a stabilizer and a histidine buffer in reducing the viscosity of a high-concentration antibody pharmaceutical formulation, or preparing a high concentration antibody pharmaceutical formulation having reduced viscosity. Preferably, the stabilizer, the histidine buffer, the antibody and the antibody concentration are as described in any one of the embodiment herein. Preferably, the use can reduce the viscosity of the high-concentration antibody pharmaceutical formulation to below about 8.0 cP (as measured at about 25° C.).

### BRIEF DESCRIPTION OF THE DRAWINGS

FIG. 1: the first round of formula screening—trend of change in the purity by SEC-HPLC in a high temperature test.

FIG. 2: the first round of formula screening—trend of change in the purity by CEX-HPLC in a high temperature test.

FIG. 3: the second round of formula screening—trend of change in the purity by SEC-HPLC in a high temperature test.

FIG. 4: the second round of formula screening—trend of change in the purity by CEX-HPLC in a high temperature test.

FIG. 5: the third round of formula screening—trend of change in the purity by SEC-HPLC in a high temperature test.

FIG. 6: the third round of formula screening—trend of change in the purity by CEX-HPLC in a high temperature test.

FIG. 7: mean plasma concentration-time curves of two groups A and B.

FIG. 8: curves of inhibitory effect of subcutaneous injection formulation on the growth of transplanted MC38 tumor in hPD-1 humanized mice.

### DETAILED DESCRIPTION

Definitions and Description In order to facilitate the understanding of the present disclosure, certain technical and scientific terms are specifically defined below. Unless otherwise specifically defined herein, all other technical and scientific terms used herein have the same meaning as commonly understood. It should be understood that the present disclosure is not limited to particular methods, agents, compounds, compositions or biological systems, which can, of course, be modified. It should also be understood that the terms used herein are for the purpose of illustrating particular embodiments only, and are not intended to be limiting. All references cited herein, including patents, patent applications, articles, textbooks and the like, and references cited therein, to the extent they have not been cited, are hereby incorporated by reference in their entirety. If one or more of the incorporated references and similar materials differ from or contradict the present application, including but not limited to defined terms, term usage, described techniques and the like, the present application controls.

As used in this specification and the appended claims, the singular forms “a”, “an” and “the” include plural referents, unless otherwise specified. Thus, for example, reference to “a polypeptide” includes a combination of two or more polypeptides and the like.

The term “pharmaceutical composition” or “formulation” means a mixture containing one or more of the antibodies described herein and other components such as physiologically acceptable carriers and excipients. The pharmaceutical composition is intended to promote the administration to an organism and facilitate the absorption of the active ingredient, to exert bioactivity.

The term “liquid formulation” refers to a formulation in liquid state and is not intended to refer to a resuspended lyophilized formulation. The liquid formulation of the present disclosure is stable during storage and its stability is independent of lyophilization (or other state change methods, such as spray drying).

The term “aqueous formulation” refers to a liquid formulation using water as the solvent. In some embodiments, the aqueous formulation is a formulation that requires no lyophilization, spray drying and/or freezing to maintain its stability (e.g., chemical and/or physical stability and/or bioactivity).

The term “excipient” refers to an agent that may be added to a formulation to provide a desired property (e.g., consistency and improved stability) and/or to adjust osmotic pressure.

Examples of commonly used excipients include, but are not limited to, sugars, polyols, amino acids, surfactants and polymers.

As used herein, when referring to measurable values (e.g., amounts and durations), “about” is intended to encompass variations of ±20% or ±10% based on the particular value, including ±5%, ±1% and ±0.1%, as such variations are suitable for implementing the disclosed methods.

The term “buffer at a pH of about 5.0-6.5” refers to an agent that, through the action of its acid/base conjugate components, renders a solution containing the agent resistant to pH changes. The buffer used in the formulation of the present disclosure may have a pH in a range of about 5.0 to about 6.5, or a pH in a range of about 5.5 to about 6.5, or a pH in a range of about 5.0 to about 6.0.

Examples of “buffer” with pH controlled in this range herein include acetic acid, acetate (e.g., sodium acetate), succinic acid, succinate (e.g., sodium succinate), gluconic acid, histidine, a histidine salt (e.g., histidine hydrochloride), methionine, citric acid, citrate, phosphate, citrate/phosphate, imidazole and a combination thereof, and other organic acid buffers.

The “histidine buffer” is a buffer containing histidine ions. Examples of histidine buffer include a buffer containing histidine and a histidine salt such as histidine hydrochloride, histidine acetate, histidine phosphate and histidine sulfate, for example, a histidine buffer containing histidine and histidine hydrochloride; the histidine buffer of the present disclosure also includes a histidine buffer containing histidine and acetate (e.g., sodium salt or potassium salt).

The “citric acid buffer” is a buffer containing citrate ions. Examples of citrate buffer include citric acid-sodium citrate, citric acid-potassium citrate, citric acid-calcium citrate, citric acid-magnesium citrate and the like. The preferred citrate buffer is a citric acid-sodium citrate buffer.

The “acetic acid buffer” is a buffer containing acetate ions. Examples of acetate buffer include acetic acid-sodium acetate, acetic acid-potassium acetate, acetic acid-calcium acetate, acetic acid-magnesium acetate and the like. The preferred acetate buffer is an acetic acid-sodium acetate buffer.

A “succinic acid buffer” is a buffer containing succinate ions. Examples of succinate buffer include succinic acid-sodium succinate, succinic acid-potassium succinate, succinic acid-calcium succinate, succinic acid-magnesium succinate and the like. The preferred succinate buffer is a succinic acid-sodium succinate buffer.

The term “stabilizer” refers to a pharmaceutically acceptable excipient that protects the active pharmaceutical ingredient and/or formulation from chemical and/or physical degradation during manufacture, storage and use. The stabilizers include, but are not limited to, sugars, amino acids, salts, polyols and metabolites thereof as defined below, such as sodium chloride, calcium chloride, magnesium chloride, mannitol, sorbitol, sucrose, trehalose, arginine or salts thereof (e.g., arginine hydrochloride), glycine, alanine (α-alanine, β-alanine), betaine, leucine, lysine, glutamic acid, aspartic acid, proline, 4-hydroxyproline, sarcosine, γ-aminobutyric acid (GABA), opines (alanopine, octopine, strombine), trimethylamine N-oxide (TMAO), human serum albumin (HSA), bovine serum albumin (BSA), α-casein, globulin, α-lactalbumin, LDH, lysozyme, myoglobin, ovalbumin and RNAase A. Some stabilizers, such as sodium chloride, calcium chloride, magnesium chloride, mannitol, sorbitol and sucrose, may also serve to control osmotic pressure. The stabilizer specifically used in the present disclosure is selected from one or more of polyols, amino acids, salts and sugars. The preferred salts are sodium chloride, the preferred sugars are sucrose and trehalose, and the preferred polyols are sorbitol and mannitol.

The preferred amino acids are arginine, glycine and proline, and the amino acids may be present in their D- and/or L-forms, but typically in the L-form, and may be present in any suitable salt forms, such as hydrochloride salts, e.g., arginine hydrochloride. The preferred stabilizers are sodium chloride, mannitol, sorbitol, sucrose, trehalose, arginine hydrochloride, glycine, proline, sodium chloride-sorbitol, sodium chloride-mannitol, sodium chloride-sucrose, sodium chloride-trehalose, arginine hydrochloride-mannitol and arginine hydrochloride-sucrose.

The term “surfactant” generally includes agents that protect proteins, such as antibodies, from air/solution interface-induced stress and solution/surface-induced stress to reduce aggregation of the antibodies or minimize the formation of particles in the formulation. Exemplary surfactants include, but are not limited to, nonionic surfactants such as polyoxyethylene sorbitan fatty acid esters (e.g., polysorbate 20 and polysorbate 80), polyethylene-polypropylene copolymers, polyethylene-polypropylene glycol, polyoxyethylene stearate, polyoxyethylene alkyl ethers (e.g., polyoxyethylene monolauryl ether), alkylphenyl polyoxyethylene ether (Triton-X), polyoxyethylene-polyoxypropylene copolymers (poloxamers, Pluronics), and sodium dodecyl sulfate (SDS). Unless otherwise specified herein, the terms “concentration of polysorbate 20” and “concentration of polysorbate 80” both refer to mass/volume concentration (w/v), for example, “0.04%” in “about 0.04% polysorbate 80” means that “100 mL of liquid contains 0.04 g of polysorbate 80”.

The term “viscosity” as used herein may be “kinematic viscosity” or “absolute viscosity”. “Kinematic viscosity” is a measure of the resistant flow of a fluid generated under the influence of gravity. “Absolute viscosity”, sometimes referred to as dynamic viscosity or simple viscosity, is the product of kinematic viscosity and fluid density (absolute viscosity=kinematic viscosity×density). The dimension of the kinematic viscosity is L²/T, where L is the length and T is the time. Generally, the kinematic viscosity is expressed in centistoke (cSt). The SI unit of the kinematic viscosity is mm²/s, i.e., 1 cSt. The absolute viscosity is expressed in centipoise (cP). The SI unit of the absolute viscosity is millipascal·second (mPa·s), where 1 cP=1 mPa·s.

For the liquid formulation of the present disclosure, the term “low viscosity” as used herein shall refer to an absolute viscosity less than about 15 centipoise (cP). For example, the liquid formulation of the present disclosure will be considered having a “low viscosity” if the formulation exhibits an absolute viscosity of about 15 cP, about 14 cP, about 13 cP, about 12 cP, about 11 cP, about 10 cP, about 9 cP, about 8 cP or less as measured by standard viscosity measurement techniques. For the liquid formulation of the present disclosure, the term “moderate viscosity” as used herein shall refer to an absolute viscosity between about 35 cP and about 15 cP. For example, the liquid formulation of the present disclosure will be considered having a “moderate viscosity” if the formulation exhibits an absolute viscosity of about 34 cP, about 33 cP, about 32 cP, about 31 cP, about 30 cP, about 29 cP, about 28 cP, about 27 cP, about 26 cP, about 25 cP, about 24 cP, about 23 cP, about 22 cP, about 21 cP, about 20 cP, about 19 cP, about 18 cP, about 17 cP, about 16 cP or about 15 cP as measured by standard viscosity measurement techniques. In some embodiments, the pharmaceutical composition of the present disclosure may exhibit an ultra-low viscosity of about 7 cP or less. In some embodiments, the viscosity, stability and efficacy of arginine or a salt thereof are found to be significantly better than those of other excipients when the viscosities of the different excipients are compared. In some embodiments, the viscosity, stability and efficacy of histidine buffer systems are found to be significantly better than those of other buffer systems when the buffer systems are compared.

The term “isotonic” refers to a formulation having substantially equivalent osmotic pressure to human blood. An isotonic formulation generally has an osmotic pressure of about 250 to 350 mOsm. Isotonicity can be measured by a vapor pressure osmometer or cryoscopic osmometer.

The term “stable” formulation is a formulation in which the antibody substantially retains its physical and/or chemical stability and/or bioactivity during the manufacture and/or storage. A pharmaceutical formulation may be considered stable even if the contained antibody fails to retain 100% of its chemical structure or biological function after a certain period of storage. In certain instances, a pharmaceutical formulation may also be considered “stable” if the contained antibody can retain about 90%, about 95%, about 96%, about 97%, about 98%, or about 99% of its structure or function after a certain period of storage. Various analytical techniques for measuring protein stability are available in the art and are described in Peptide and Protein Drug Delivery, 247-301, Vincent Lee ed., Marcel Dekker, Inc., New York, N.Y., Pubs. (1991) and Jones, A., (1993) Adv. Drug Delivery Rev., 10: 29-90, both of which are incorporated herein for reference.

After a certain period of storage at a certain temperature, the stability of the formulation can be measured by determining the percentage of remaining natural antibody (and by other methods). Except other methods, the percentage of natural antibody can be measured by size-exclusion chromatography (e.g., size-exclusion high-performance liquid chromatography (SEC-HPLC)), and “natural” refers to unaggregated and undegraded. In some embodiments, the stability of a protein is determined by a percentage of monomeric protein in a solution having a low percentage of degraded (e.g., fragmented) and/or aggregated protein. In some embodiments, the formulation can be stably stored at room temperature, about 25-30° C., or 40° C. for at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months or longer, and has no more than about 6%, 5%, 4%, 3%, 2%, 1%, 0.5%, or 0.1% antibody in aggregated form.

Stability can be measured by determining the percentage of antibody that migrates in a fraction that is more acidic (“acidic form”) than the main fraction of the antibody (“mainly charged form”) during ion exchange (and by other methods), and the stability is inversely proportional to the percentage of antibodies in the acidic form. The percentage of “acidified” antibody can be measured by, except other methods, ion exchange chromatography (e.g., cation exchange high-performance liquid chromatography [CEX-HPLC]). In some embodiments, an acceptable stability means that the antibodies in the acidic form that can be detected in the formulation is no more than about 49%, 45%, 40%, 35%, 30%, 25%, 20%, 15%, 10%, 5%, 4%, 3%, 2%, 1%, 0.5% or 0.1% after a certain period of storage at a certain temperature. The certain period of storage prior to measuring stability can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or longer. When the stability is assessed, the certain temperature at which the pharmaceutical formulation is allowed to be stored may be any temperature in a range of about −80° C. to about 45° C., e.g., about −80° C., about −30° C., about −20° C., about 0° C., about 2-8° C., about 5° C., about 25° C. or about 40° C.

An antibody in pharmaceutical composition “retains its physical stability” if the antibody does not substantially show signs of, such as aggregation, precipitation and/or denaturation, during visual inspection of color and/or clarity or when measured by UV light scattering or pore-exclusion chromatography. Aggregation is a process in which individual molecules or complexes associate, covalently or non-covalently, to form aggregates. Aggregation can proceed to the point where a visible precipitate is formed.

Stability, e.g., physical stability, of a formulation can be assessed by methods well known in the art, including measuring the apparent extinction (absorbance or optical density) of a sample. Such extinction measurement correlates with the turbidity of the formulation. Turbidity of a formulation is, in part, an inherent property of proteins dissolved in solution and is generally measured by nephelometry and is measured in a nephelometric turbidity unit (NTU).

Turbidity levels that vary with, for example, the concentration of one or more components in a solution (e.g., protein and/or salt concentration) are also referred to “opacification” or “opacified appearance” of a formulation. Turbidity levels can be calculated with reference to a standard curve based on suspensions of known turbidity. The reference standards for determining the turbidity level of a pharmaceutical composition may be based on the standard of the European Pharmacopoeia, 4^{th} edition, Directorate for the Quality of Medicine of the Council of Europe (EDQM), Strasbourg, France. According to the standard of European Pharmacopoeia, a clear solution is defined as a solution having a turbidity lower than or equal to that of a reference suspension having a turbidity of about 3 according to the standard of European Pharmacopoeia. Turbidity measurement by nephelometry can measure Rayleigh scattering in the absence of association or non-ideal effects, which generally varies linearly with concentration. Other methods for assessing physical stability are well known in the art.

An antibody “retains its chemical stability” in a pharmaceutical composition if the chemical stability of the antibody at a given time point allows the antibody to retain its bioactivity as defined hereinafter. Chemical stability can be assessed, for example, by detecting or quantifying the form of chemical changes in the antibody. Chemical changes can include size changes (e.g., truncation), which can be assessed by, for example, size-exclusion chromatography, SDS-PAGE, and/or matrix-assisted laser desorption/ionisation time-of-flight mass spectrometry (MALDI/TOF MS). Other types of chemical changes include charge changes (e.g., occurring as a result of deamidation or oxidation), which can be assessed by, for example, ion exchange chromatography.

An antibody in a pharmaceutical composition “retains its bioactivity” if it is biologically active for its intended purpose. For example, the formulation described herein is considered stable if, after a certain period of storage (e.g., 1 to 12 months) at a certain temperature, e.g., 5° C., 25° C. or 45° C., the binding affinity of the anti-PD-1 antibody in the formulation for PD-1 is at least 90%, 95% or greater of that of the antibody prior to storage. Binding affinity can also be determined by, for example, ELISA or plasmon resonance.

In the context of the present disclosure, a “therapeutically effective amount” or “effective amount” of an antibody, pharmacologically refers to an amount that is effective in preventing, treating or alleviating the symptoms of a disorder that the antibody can effectively treat. In the present disclosure, a “therapeutically effective amount” or “therapeutically effective dose” of a medicament is any amount of the medicament that, when used alone or in combination with an additional therapeutic agent, protects a subject from the onset of a disease or promotes the regression of a disease as evidenced by a decrease in the severity of disease symptoms, an increase in the frequency and duration of disease symptom-free phase, or the prevention of injury or disability resulting from the affliction of the disease. The ability of a medicament to promote the regression of a disease can be assessed using a variety of methods, such as in human subjects during clinical trials, in animal model systems that predict human efficacy, or by determining the activity of the medicament in an in vitro assay. A therapeutically effective amount of a medicament includes a “prophylactically effective amount” which is any amount of a medicament that, when administered alone or in combination with other therapeutic drugs to a subject at risk of developing diseases or a subject having disease recurrence, inhibits the development or recurrence of diseases.

The term “subject” or “patient” is intended to include mammalian organisms. Examples of subjects/patients include human and non-human mammals such as non-human primates, dogs, cows, horses, pigs, sheep, goats, cats, mice, rabbits, rats and transgenic non-human animals. In one specific embodiment of the present disclosure, the subject is human.

The terms “administering”, “giving” and “treating” refer to introducing a composition including a therapeutic agent into a subject using any one of a variety of methods or delivery systems. Routes of administration of the anti-PD-1 antibody include intravenous, intramuscular, subcutaneous, intraperitoneal, spinal or other parenteral routes of administration, such as injection or infusion. “Parenteral administration” refers to modes of administration apart from enteral or local administration, typically by injection, including but not limited to, intravenous, intramuscular, intra-arterial, intrathecal, intralymphatic, intralesional, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion, and in vivo electroporation.

### Anti-PD-1 Antibody

The term “antibody” as used herein should be construed as including intact antibody molecules and antigen-binding fragments thereof. The term “antigen-binding moiety” or “antigen-binding fragment” (or simply “antibody moiety” or “antibody fragment”) of an antibody, as used herein, refers to one or more fragments of an antibody that retain the ability to specifically bind to human PD-1 or an epitope thereof. Thus, it is used in the broadest sense and specifically includes, but is not limited to, monoclonal antibodies (including full-length monoclonal antibodies), polyclonal antibodies, multispecific antibodies (e.g., bispecific antibodies), humanized antibodies, fully human antibodies, chimeric antibodies, and camelized single-domain antibodies.

An “isolated antibody” refers to the purified state of a binding compound, and, in this case, means that the molecule is substantially free of other biomolecules, such as nucleic acids, proteins, lipids, sugars, or other substances such as cell debris and growth medium. The term “isolate(d)” does not mean the complete absence of such substances or the absence of water, buffers or salts, unless they are present in amounts that will significantly interfere with the experimental or therapeutic use of the binding compounds described herein.

A “monoclonal antibody” refers to an antibody obtained from a substantially homogeneous population of antibodies, i.e., the antibodies composing the population are identical except for possible naturally occurring mutations that may be present in minor amounts. A monoclonal antibody is highly specific and targets a single antigen epitope. In contrast, conventional (polyclonal) antibody preparations typically include a large number of antibodies targeting (or specific for) different epitopes. The modifier “monoclonal” indicates the characteristic of an antibody obtained from a substantially homogeneous population of antibodies, and is not to be construed as producing the antibody by any particular method.

The term “murine antibody” or “hybridoma antibody” in the present disclosure refers to an anti-human PD-1 monoclonal antibody prepared according to the knowledge and skills in the art. The preparation is carried out by injecting the test subject with the PD-1 antigen and then isolating hybridomas expressing antibodies with the desired sequences or functional properties.

A “chimeric antibody” is an antibody having the variable domains of a first antibody and the constant domains of a second antibody, and the first and second antibodies are from different species. Typically, the variable domains are obtained from an antibody of an experimental animal such as a rodent (“parent antibody”), and the constant domain sequences are obtained from a human antibody, and the resulting chimeric antibody is less likely to induce an adverse immune response in a human subject as compared to the parent rodent antibody.

A “humanized antibody” refers to an antibody form containing sequences from both human and non-human (such as mouse and rat) antibodies. In general, a humanized antibody includes substantially all of at least one, and typically two, variable domains, in which all or substantially all of the hypervariable loops correspond to those of a non-human immunoglobulin and all or substantially all of the framework regions (FRs) are those of a human immunoglobulin sequence. The humanized antibody may include at least a portion of a human immunoglobulin constant region (Fc).

The term “full-length antibody” or “intact antibody molecule” refers to an immunoglobulin molecule including four peptide chains, including two heavy (H) chains (about 50-70 kDa in total length) and two light (L) chains (about 25 kDa in total length) linked to each other by disulfide bonds. Each heavy chain consists of a heavy chain variable region (abbreviated herein as VH) and a heavy chain constant region (abbreviated herein as CH). The heavy chain constant region consists of 3 domains (CH1, CH2 and CH3). Each light chain consists of a light chain variable region (abbreviated herein as VL) and a light chain constant region. The light chain constant region consists of one domain (CL). The VH and VL regions can be further divided into complementarity determining regions (CDRs) with high variability and more conservative regions called framework regions (FRs) that are spaced apart by the CDRs. Each VH or VL region consists of 3 CDRs and 4 FRs arranged in the following order from the amino terminus to the carboxyl terminus: FR1, CDR1, FR2, CDR2, FR3, CDR3, FR4. The variable regions of the heavy and light chains contain binding domains that interact with antigens. The constant regions of an antibody can mediate the binding of immunoglobulins to host tissues or factors, including various cells of the immune system (e.g., effector cells) and the first component (Clq) of classical complement system.

The term “CDR” refers to a complementarity determining region within an antibody variable sequence. There are 3 CDRs in each of the heavy chain and light chain variable regions, which are named HCDR1, HCDR2 and HCDR3 for the heavy chain variable region, or LCDR1, LCDR2 and LCDR3 for the light chain variable region. Exact boundaries of the CDRs may vary among different systems.

The precise amino acid sequence boundaries of the variable region CDRs of the antibodies of the present disclosure can be determined using any of a number of well-known schemes, including Chothia based on the three-dimensional structure of antibodies and the topology of the CDR loops (Chothia et al., (1989) Nature 342: 877-883; Al-Lazikani et al., Standard conformations for the canonical structures of immunoglobulins, Journal of Molecular Biology, 273, 927-948 (1997)), Kabat based on antibody sequence variability (Kabat et al., Sequences of Proteins of Immunological Interest, 4^{th} edition, U.S. Department of Health and Human Services, National Institutes of Health (1987)), AbM (University of Bath), Contact (University College London), International ImMunoGeneTics database (IMGT) (1999 Nucleic Acids Research, 27, 209-212), and North CDR definition based on the affinity propagation clustering using a large number of crystal structures. The boundaries of the CDRs of the antibodies of the present disclosure can be determined according to any scheme (e.g., different assignment systems or combinations) in the art.

An “antigen-binding fragment” as used herein includes an antibody fragment or a derivative thereof, generally including at least one fragment of an antigen-binding region or variable region (e.g., one or more CDRs) of a parent antibody, which retains at least some of the binding specificity of the parent antibody. Examples of antigen-binding fragments include, but are not limited to, Fab, Fab′, F(ab′)₂ and Fv fragments; a diabody; a linear antibody; a single-chain antibody molecule, such as sc-Fv; a nanobody and multispecific antibody formed by antibody fragments. A binding fragment or a derivative thereof generally retains at least 10% of the antigen-binding activity of the parent antibody when the binding activity of the antibody is expressed on a molar concentration basis. Preferably, the binding fragment or the derivative thereof retains at least 20%, 50%, 70%, 80%, 90%, 95% or 100% or more of the antigen-binding affinity of the parent antibody. It is also contemplated that an antigen-binding fragment of an antibody may include conservative or non-conservative amino acid substitutions that do not significantly alter its bioactivity (referred to as “conservative variants” or “function-conservative variants” of the antibody).

The anti-PD-1 antibody or the antigen-binding fragment thereof described herein includes any one of anti-PD-1 antibodies or antigen-binding fragments thereof described in application No. CN201310258289.2, which is incorporated herein by reference in its entirety. In some embodiments, the CDR sequences of the antibody used in the method and composition of the present disclosure include the CDR sequences from humanized antibody clone 38 described in CN201310258289.2.

In some embodiments, the anti-PD-1 antibody or the antigen-binding fragment thereof used in the method and composition of the present disclosure includes an LCDR1, an LCDR2 and an LCDR3 having amino acid sequences set forth in SEQ ID NO: 1, SEQ ID NO: 2 and SEQ ID NO: 3, respectively, and an HCDR1, an HCDR2 and an HCDR3 having amino acid sequences set forth in SEQ ID NO: 4, SEQ ID NO: 5 and SEQ ID NO: 6, respectively.

In some embodiments, the anti-PD-1 antibody or the antigen-binding fragment thereof used in the method and composition of the present disclosure is selected from a murine antibody or an antigen-binding fragment thereof, a chimeric antibody or an antigen-binding fragment thereof, and a humanized antibody or an antigen-binding fragment thereof, preferably a humanized antibody or an antigen-binding fragment thereof.

In some embodiments, the anti-PD-1 antibody or the antigen-binding fragment thereof used in the method and composition of the present disclosure includes a light chain variable region set forth in SEQ ID NO: 7 and a heavy chain variable region set forth in SEQ ID NO: 8.

In some embodiments, the anti-PD-1 antibody used in the method and composition of the present disclosure includes a light chain amino acid sequence set forth in SEQ ID NO: 9 and a heavy chain amino acid sequence set forth in SEQ ID NO: 10.

In some embodiments, the non-limiting and exemplary antibody used in the examples herein is toripalimab (a humanized IgG4 mAb having the structure described in WHO Drug Information, 32(2), 372-373 (2018) and including the heavy and light chain amino acid sequences set forth in SEQ ID NOs: 9 and 10, respectively).

| SEQ ID NO: | Amino Acid Sequence |
|---|---|
| 1 | RSSQSIVHSNGNTYLE |
| 2 | KVSNRFS |
| 3 | FQGSHVPLT |
| 4 | DYEMH |
| 5 | VIESETGGTAYNQKFKG |
| 6 | EGITTVATTYYWYFDV |
| 7 | DVVMTQSPLSLPVTLGQPASISCRSSQSIVHSNGNTYLEWYLQKPGQ |
| | SPQLLIYKVSNRFSGVPDRFSGSGSGTDFTLKISRVEAEDVGVYYCF |
| | QGSHVPLTFGQGTKLEIK |
| 8 | QGQLVQSGAEVKKPGASVKVSCKASGYTFTDYEMHWVRQAPIHG |
| | LEWIGVIESETGGTAYNQKFKGRVTITADKSTSTAYMELSSLRSEDT |
| | AVYYCAREGITTVATTYYWYFDVWGQGTTVTVSS |
| 9 | DVVMTQSPLSLPVTLGQPASISCRSSQSIVHSNGNTYLEWYLQKPGQ |
| | SPQLLIYKVSNRFSGVPDRFSGSGSGTDFTLKISRVEAEDVGVYYCF |
| | QGSHVPLTFGQGTKLEIKRTVAAPSVFIFPPSDEQLKSGTASVVCLL |
| | NNFYPREAKVQWKVDNALQSGNSQESVTEQDSKDSTYSLSSTLTLS |
| | KADYEKHKVYACEVTHQGLSSPVTKSFNRGEC |
| 10 | QGQLVQSGAEVKKPGASVKVSCKASGYTFTDYEMHWVRQAPIHG |
| | LEWIGVIESETGGTAYNQKFKGRVTITADKSTSTAYMELSSLRSEDT |
| | AVYYCAREGITTVATTYYWYFDVWGQGTTVTVSSASTKGPSVFPL |
| | APCSRSTSESTAALGCLVKDYFPEPVTVSWNSGALTSGVHTFPAVL |
| | QSSGLYSLSSVVTVPSSSLGTKTYTCNVDHKPSNTKVDKRVESKYG |
| | PPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSR |
| | LTVDKSRWQEGNVFSCSVMHEALHNHYTQKSLSLSLGK |

In some embodiments, the anti-PD-i antibody or the antigen-binding fragment thereof used in the method and composition of the present disclosure is a humanized or chimeric antibody, and may include human constant regions. In some embodiments, the constant region is selected from human IgG1, IgG2, IgG3 and IgG4 constant regions; preferably, the anti-PD-i antibody or the antigen-binding fragment thereof suitable for use in the method and composition described herein includes a heavy chain constant region of human IgG1 or IgG4 isotype, more preferably a human IgG4 constant region. In some embodiments, the sequence of the IgG4 heavy chain constant region of the anti-PD-i antibody or the antigen-binding fragment thereof includes the S228P mutation that replaces a serine residue in the hinge region with a proline residue that is typically present at the corresponding position of an antibody of IgGI isotype.

In some embodiments, the present disclosure provides a method for preparing the anti-PD-1 antibody or the antigen-binding fragment thereof described herein, which includes expressing the antibody or the antigen-binding fragment thereof in the host cell described herein under conditions suitable for expression of the antibody or the antigen-binding fragment thereof, and isolating the expressed antibody or antigen-binding fragment thereof from the host cell.

The present disclosure provides a mammalian host cell for expressing the recombinant antibody of the present disclosure, which includes a number of immortalized cell lines available from American Type Culture Collection (ATCC). Those cell lines include, in particular, Chinese hamster ovary (CHO) cells, NS0, SP2/0 cells, HeLa cells, baby hamster kidney (BHK) cells, monkey kidney cells (COS), human hepatocellular carcinoma cells, A549 cells, 293T cells and many other cell lines. Mammalian host cells include human, mouse, rat, dog, monkey, pig, goat, cow, horse and hamster cells. Particularly preferred cell lines are selected by determining which cell line has high expression level.

In one embodiment, the present disclosure provides a method for preparing an anti-PD-1 antibody, which includes: introducing an expression vector into a mammalian host cell, and culturing the host cell for a period of time sufficient to allow expression of the antibody in the host cell or more preferably to allow secretion of the antibody into a medium in which the host cell is grown, to produce the antibody. The antibody can be isolated from the medium using standard protein purification methods.

It is likely that antibodies expressed by different cell lines or in transgenic animals have different glycosylations from each other. However, all antibodies encoded by the nucleic acid molecules provided herein or including the amino acid sequences provided herein are integral parts of the present disclosure, regardless of the glycosylation of the antibody. Likewise, in some embodiments, nonfucosylated antibodies are advantageous because they generally have more potent efficacy in vitro and in vivo than their fucosylated counterparts, and are unlikely to be immunogenic because their glycan structures are normal components of natural human serum IgG.

### Pharmaceutical Formulation

The pharmaceutical composition described herein is a pharmaceutical composition with high concentration, high stability and ultra-low viscosity comprising an antibody specifically binding to PD-1. The clinical need for subcutaneous (SC) administration of protein drugs at a high dose of >100 mg/mL often introduces additional development challenges with respect to manufacturing, analytical testing, stability and delivery. A common property of high-concentration protein formulation is high viscosity, which is directly caused by the reversible self-association of the protein. High viscosity can also pose additional clinical development challenges due to high injection force (increased pain at the injection site) and can also alter the pharmacokinetic properties of the drug. Therefore, an important factor of product development efforts is the search and identification of formulations with low viscosity. The present disclosure develops a high-concentration antibody formulation by selecting a suitable buffer system and pH, optimizing stabilizer and surfactant, and carrying out pharmacokinetics and pharmacodynamics studies, which can be used for subcutaneous administration dosage forms, and has long-term stability, no aggregation and ultra-low viscosity.

The present disclosure provides a pharmaceutical composition including: (1) a buffer; and (2) an anti-PD-1 antibody or an antigen-binding fragment thereof.

The anti-PD-1 antibody or the antigen-binding fragment thereof in the pharmaceutical composition described herein is as described in any one of the embodiments of the “Anti-PD-1 Antibody” section of the present application.

For example, the anti-PD-1 antibody or the antigen-binding fragment thereof in the pharmaceutical composition described herein includes an LCDR1, an LCDR2 and an LCDR3 having amino acid sequences set forth in SEQ ID NO: 1, SEQ ID NO: 2 and SEQ ID NO: 3, respectively, and an HCDR1, an HCDR2 and an HCDR3 having amino acid sequences set forth in SEQ ID NO: 4, SEQ ID NO: 5 and SEQ ID NO: 6, respectively. Preferably, the anti-PD-1 antibody or the antigen-binding fragment thereof is selected from a murine antibody or an antigen-binding fragment thereof, a chimeric antibody or an antigen-binding fragment thereof, and a humanized antibody or an antigen-binding fragment thereof, preferably a humanized antibody or an antigen-binding fragment thereof. Preferably, the anti-PD-1 antibody or the antigen-binding fragment thereof comprises a light chain variable region set forth in SEQ ID NO: 7 and a heavy chain variable region set forth in SEQ ID NO: 8. More preferably, the anti-PD-1 antibody includes a light chain amino acid sequence set forth in SEQ ID NO: 9 and a heavy chain amino acid sequence set forth in SEQ ID NO: 10.

The anti-PD-1 antibody or the antigen-binding fragment thereof in the pharmaceutical composition described herein has a concentration of about 100-250 mg/mL, preferably about 150-250 mg/mL, and more preferably about 150-200 mg/mL.

The pharmaceutical composition described herein has a pH of about 5.0-6.5, preferably about 5.5-6.2, and more preferably about 6.0.

The buffer in the pharmaceutical composition described herein is selected from one or more of an acetic acid buffer, a citric acid buffer and a histidine buffer, preferably a histidine buffer. Preferably, the histidine buffer is selected from a histidine-histidine hydrochloride buffer and a histidine-histidine acetate buffer, preferably a histidine-histidine hydrochloride buffer. Preferably, the buffer has a concentration of about 5-100 mM, preferably about 10-50 mM, preferably about 10-30 mM, and preferably about 15-25 mM. Preferably, the buffer has a pH of about 5.0-6.5, preferably about 5.5-6.5, and preferably about 5.5-6.2.

Accordingly, the pharmaceutical composition of the present disclosure may include: a histidine-histidine hydrochloride buffer having a pH of about 5.5-6.5 and a concentration of about 10-30 mM in the pharmaceutical composition; and about 150-250 mg/mL anti-PD-1 antibody or antigen-binding fragment thereof described in any one of the foregoing embodiments, particularly the humanized antibody clone 38 or the antigen-binding fragment thereof described herein.

In some embodiments, the pharmaceutical composition described herein further includes a stabilizer selected from one or more of arginine, an arginine salt, sodium chloride, mannitol, sorbitol, sucrose, glycine and trehalose; preferably, the arginine salt is arginine hydrochloride. Preferably, the stabilizer has a concentration of about 10-400 mM, preferably about 100-250 mM, preferably about 120-220 mM, and preferably about 130-180 mM. Preferably, the stabilizer is arginine or an arginine salt having a concentration of about 120-220 mM; or the stabilizer is a combination of arginine hydrochloride having a concentration of about 30-100 mM and sucrose having a concentration of about 100-180 mM; or the stabilizer is a combination of arginine hydrochloride having a concentration of about 30-100 mM and glycine having a concentration of about 50-150 mM; preferably, the stabilizer is arginine or an arginine salt having a concentration of about 130-180 mM; or the stabilizer is a combination of arginine hydrochloride having a concentration of about 30-70 mM and sucrose having a concentration of about 110-170 mM; or the stabilizer is a combination of arginine hydrochloride having a concentration of about 30-70 mM and glycine having a concentration of about 80-120 mM; preferably, the arginine salt is arginine hydrochloride.

Accordingly, the pharmaceutical composition of the present disclosure may include: a histidine-histidine hydrochloride buffer having a pH of about 5.5-6.5 and a concentration of about 10-30 mM in the pharmaceutical composition; about 150-250 mg/mL anti-PD-1 antibody or antigen-binding fragment thereof described in any one of the foregoing embodiments, particularly the humanized antibody clone 38 or the antigen-binding fragment thereof described herein; and about 100-250 mM stabilizer, preferably, the stabilizer is selected from one or more of arginine, an arginine salt, sodium chloride, mannitol, sorbitol, sucrose, glycine and trehalose, and preferably, the arginine salt is arginine hydrochloride. Preferably, the stabilizer is arginine or an arginine salt having a concentration of about 120-220 mM; or the stabilizer is a combination of arginine hydrochloride having a concentration of about 30-100 mM and sucrose having a concentration of about 100-180 mM; or the stabilizer is a combination of arginine hydrochloride having a concentration of about 30-100 mM and glycine having a concentration of about 50-150 mM.

In some embodiments, the pharmaceutical composition further includes a surfactant selected from one or more of polysorbate 80, polysorbate 20 and poloxamer 188. Preferably, based on w/v, the surfactant has a concentration of about 0.001%-0.1%, preferably about 0.01%-0.1%, and preferably about 0.02%-0.08%.

Accordingly, the pharmaceutical composition of the present disclosure may include: a histidine-histidine hydrochloride buffer having a pH of about 5.5-6.5 and a concentration of about 10-30 mM in the pharmaceutical composition; about 150-250 mg/mL anti-PD-1 antibody or antigen-binding fragment thereof described in any one of the foregoing embodiments, particularly the humanized antibody clone 38 or the antigen-binding fragment thereof described herein; about 100-250 mM stabilizer, preferably, the stabilizer is arginine or an arginine salt having a concentration of about 120-220 mM, or the stabilizer is a combination of arginine hydrochloride having a concentration of about 30-100 mM and sucrose having a concentration of about 100-180 mM, or the stabilizer is a combination of arginine hydrochloride having a concentration of about 30-100 mM and glycine having a concentration of about 50-150 mM; and based on w/v, about 0.01%-0.1% polysorbate 80.

The pharmaceutical composition of the present disclosure is a liquid formulation or a lyophilized formulation.

The pharmaceutical composition has an osmotic pressure in a range of 260-320 mOsm/kg, preferably in a range of 290-310 mOsm/kg.

The pharmaceutical composition of the present disclosure has a viscosity of ≤8.0 cP as measured at about 25° C.

### Pharmaceutical Use and Method

The present disclosure further provides use of the pharmaceutical composition or the injection described in any one of the embodiments herein in preparing a drug for treating a disease or a disorder by eliminating, inhibiting or reducing PD-1 activity.

The present disclosure further provides the pharmaceutical composition or the injection described in any one of the embodiments herein for treating a disease or a disorder by eliminating, inhibiting or reducing PD-1 activity.

The present disclosure further provides a method for treating a disease or a disorder by eliminating, inhibiting or reducing PD-1 activity, which includes administering to a subject in need thereof the pharmaceutical composition or the injection described in any one of the embodiments herein.

In some embodiments, the disease or disorder is selected from cancer, infectious diseases and inflammatory diseases; preferably, the cancer is selected from colon cancer, neuroendocrine neoplasm, esophageal cancer, nasopharyngeal cancer, sarcoma, melanoma, urothelial cancer and non-small cell lung cancer.

### EXAMPLES

The present disclosure will be illustrated hereinafter by way of specific examples. It should be understood that these examples are illustrative only and are not intended to limit the scope of the present disclosure. The present disclosure has been described in detail, and the specific embodiments are also disclosed. It will be apparent that various changes and improvements can be made in the specific embodiments of the present disclosure without departing from the spirit and scope of the present disclosure, and any modifications, equivalents, improvements, etc., are intended to be included within the scope of the present disclosure. The methods and materials used in the examples are, unless otherwise indicated, conventional in the art.

The detection method used in the examples includes:

### (1) Appearance

Appearance was determined by visual inspection. The illumination intensity of the clarity detector was ensured to be kept between 1000 lx and 1500 lx. The sample was kept at the same level as the eye, and gently shaken or inverted to avoid bubbles. Visual inspection was performed in front of black background and white background. The results were recorded in terms of three aspects of color, opalescence and visible particles.

### (2) Protein Content

Protein concentration was determined using Nanodrop and SoloVPE. When the Nanodrop was used, the percent extinction coefficient (E1%) was set at 1.416 (mg/mL)⁻¹cm⁻¹. The detector was washed three times with ultrapure water, 3 μL of ultrapure water was added to the detection well, the “calibration” button was clicked and the calibration was performed by taking the ultrapure water as a blank. After blank calibration, the sample was determined. 3 μL of sample was added to the detection well, the “test” button was clicked, and the test data were recorded. Three solutions of each sample were determined in parallel, and each solution was determined once.

When the SoloVPE was used for determination, the percent extinction coefficient (E1%) was set at 1.416 (mg/mL)⁻¹cm⁻¹. After calibration with the ultrapure water as a blank, the sample was measured. 120 μL of the sample was added to the cuvette, the “test” button was clicked, and the test data were recorded.

### (3) Purity by SEC-HPLC

The purity by SEC-HPLC was determined by HPLC (Waters e2695 instrument) equipped with an SEC column (TSK gel G3000SWXL, 7.8×300 mm, 5 μm). The mobile phase consists of 50 mM phosphate and 300 mM Na₂SO₄, pH 7.0±0.2. The results were quantitatively analyzed by peak area normalization. The peak area percentages of the monomer, the polymer and the fragment were calculated, respectively. The peak area percentage of the monomer was taken as the purity of the sample, and the peak area percentages of the polymer and the fragment were taken as the content of the polymer and the fragment. The chromatographic parameters are shown in Table 1 below.

TABLE 1
SEC-HPLC chromatographic parameters

| Chromatographic conditions | Chromatographic parameters | |
|---|---|---|
| Chromatographic column | TSK gel G3000SWXL, | |
| | 7.8 * 300 mm, 5 μm | |
| Wavelength of detector | 280 | nm |
| Temperature of autosampler | 5 ± 3° | C. |
| Temperature of column | 25 ± 2° | C. |
| Flow rate | 0.5 | mL/min |
| Injection volume | 25 | μL |
| Operation mode | Isocratic elution | |
| Elution time | 30 | min |

### (4) Purity by R-CE-SDS

The determination of purity of the antibody formulation by the reduced CE-SDS electrophoresis was performed by taking a high-voltage direct-current electric field as driving force and taking a capillary as a separation channel. The pre-filled gel can form a molecular sieve in the capillary, sodium dodecyl sulfate can eliminate the charge effect of different protein molecules, and the reducing agent β-mercaptoethanol can cleave disulfide bond in sample, and the samples with different molecular sizes move at different speeds in the capillary and thus can be separated. The sample was diluted to 1 mg/mL with a loading buffer (SDS-MW sample buffer); 95 μL of loading buffer (SDS-MW sample buffer) was taken, 5 μL of β-mercaptoethanol was added, and the system was mixed well by vortex and used as blank control. 95 μL of test sample (1 mg/mL) was taken, and 5 μL of β-mercaptoethanol was added. The mixture was centrifuged at 3000 rpm at room temperature for 30 s, incubated at 70±2° C. for 15±2 min, cooled to room temperature, centrifuged at 6000 rpm at room temperature for 1 min, separated for 40 min and determined by using capillary electrophoresis apparatus (Beckman). The purity values of the heavy chain (HC), the non-glycosylated heavy chain (NGHC) and the light chain (LC) were calculated, and the sum of the three is the purity of the sample.

### (5) Purity by NR-CE-SDS

The determination of purity of the antibody formulation by the non-reduced CE-SDS electrophoresis was performed by taking a high-voltage direct-current electric field as driving force and taking a capillary as a separation channel. The pre-filled gel can form a molecular sieve in the capillary and the samples are treated with sodium dodecyl sulfate to eliminate the charge effect of different protein molecules, and the samples with different molecular sizes move at different speeds in the capillary and thus can be separated. Adding alkylating reagent to the test sample solution can reduce the component diffusion effectively, resulting in sharp peaks and high separation efficiency, and can ensure that the sample remains in a non-reduced state. The sample was diluted to 1 mg/mL with a loading buffer (SDS-MW sample suffer); 95 μL of loading buffer (SDS-MW sample buffer) was taken, and 5 μL of 0.8 M iodoacetamide solution was added, and the system was mixed well by vortex and used as blank control; 95 μL of test sample (1 mg/mL) was taken, 5 μL of 0.8 M iodoacetamide solution was added, and the mixture was centrifuged at 3000 rpm at room temperature for 30 s, incubated at 70±2° C. for 5±1 min, cooled to room temperature, centrifuged at 6000 rpm at room temperature for 1 min and determined by using capillary electrophoresis apparatus (Beckman).

### (6) Purity by CEX-HPLC

The purity by CEX-HPLC was determined by HPLC (Waters e2695 instrument) equipped with a chromatographic column (ProPac WCX-10, 4×250 mm). The mobile phase consists of: phase A: 10 mM sodium dihydrogen phosphate dihydrate solution (pH 4.7±0.2); phase B: 10 mM disodium hydrogen phosphate dodecahydrate solution (pH 9.2±0.2). The percentages of the main peak, the acidic peak and the basic peak were calculated by peak area normalization. If a reasonable integral result cannot be obtained by using automatic integration, manual integration is used. The detailed chromatographic parameters are shown in Table 2 below.

TABLE 2
CEX-HPLC chromatographic parameters

| Chromatographic conditions | Chromatographic parameters | | |
|---|---|---|---|
| Chromatographic column | ProPac WCX-10, 4*250 mm | | |
| Detection wavelength | 280 nm | | |
| Temperature of column | 30° C. | | |
| Temperature of autosampler | 5° C. | | |
| Flow rate | 1.0 mL/min | | |
| Sample concentration | 1.0 mg/mL | | |
| Injection volume | 100 μL | | |
| Mobile phase | Mobile phase A: 10 mM sodium | | |
| | dihydrogen phosphate dihydrate | | |
| | solution | | |
| | Mobile phase B: 10 mM disodium | | |
| | hydrogen phosphate dodecahydrate | | |
| | solution | | |
| Gradient elution procedure | Time (min) | A % | B % |
| | 0 | 72 | 28 |
| | 2 | 72 | 28 |
| | 35 | 38 | 62 |
| | 35.01 | 0 | 100 |
| | 40 | 0 | 100 |
| | 40.01 | 72 | 28 |
| | 60 | 72 | 28 |

### (7) Cell Viability

In this method, PD-1 Jurkat T cells were used as effector cells, and CHO engineered cells overexpressing PD-L1 were used as target cells. T cell antigen receptor on Jurkat effector cell can bind to the antigen on CHO target cell, and can inhibit the expression of NFAT luciferase reporter gene. PD-1 monoclonal antibody can bind to PD-1 on Jurkat T cell, to block PD-1 on Jurkat T cell from interacting with PD-LI on CHO target cell, promoting T cell activation, and activating NFAT luciferase reporter gene. The luciferase assay reagent was added, and the intensity of the signal generated by NFAT-luciferase expression in the Jurkat T cell was determined by the chemiluminescence method using a microplate reader to investigate the binding ability of PD-1 monoclonal antibody to PD-1 molecule.

### (8) Binding Activity

In this method, an indirect method was adopted. Human PD-1 was coated in a 96-well plate as an antigen. PD-1 monoclonal antibody can bind to human PD-1. Biotinylated antibody (biotinylated mouse anti-human IgG4) can specifically bind to PD-1 monoclonal antibody bound to solid phase antigen (human PD-1). Horseradish peroxidase-labeled streptavidin (peroxidase-conjugated streptavidin) can bind to the biotinylated antibody (biotinylated mouse anti-human IgG4). The horseradish peroxidase-labeled streptavidin can catalyze TMB to display blue under the action of hydrogen peroxide and the blue shade is in positive correlation with the bonding amount of the horseradish peroxidase-labeled streptavidin. The solution turned yellow after the reaction was stopped by 2 M hydrochloric acid. The absorbance (OD value) was determined at a wavelength of 450 nm/620 nm using a microplate reader, and the effective binding activity (EC₅₀) was determined according to the standard curve, to investigate the binding ability of PD-1 monoclonal antibody to PD-1.

### (9) Sub-Visible Particle Detection by MFI

The sub-visible particle detection by MFI was performed by using a particle detector (MFI5100). Due to the high concentration of the sample, the sample needs to be diluted before loading. After dilution, the system was gently and fully mixed to avoid bubbles, and 1.3 mL of the sample was pipetted into a loading plate using a pyrogen-free pipette tip in the super-clean bench. The loading plate was covered tightly with clean tin foil, and moved to the corresponding working plate of the instrument from the super-clean bench. The loading position was input into the instrument, and the sequence detection was performed. When the sample flowed through the flow cell, the sub-visible particles were photographed by a tiny camera and counted.

### (10) Sub-Visible Particle Detection by HIAC

The sub-visible particle detection was performed by HIAC. The sample was gently and fully mixed to avoid bubbles and then placed in a sample well. The sample was taken automatically by a mechanical arm of the instrument. When the sample flowed through the sensor, the sub-visible particles were counted by the light-blockage method.

### (11) Viscosity

The viscosity was determined using a viscometer (manufacturer: RheoSense, model: MicroVisc) at a determination temperature of about 25° C. and a shear rate of about 1000-2000 s⁻¹.

Abbreviations in the following examples: “hr” represents hour, “W” represents week, “M” represents month, “C” represents the number of freezing/thawing cycles, “FT” represents freezing/thawing cycle, “RT” represents room temperature, and “TO” represents the initial test of the formula samples prior to the storage.

### Example 1. Preliminary Screening for Buffer System and Stabilizer

In a liquid pharmaceutical composition, the buffer system and pH closely affect the stability of the antibody, and each antibody with unique physicochemical properties has an optimum buffer and pH. This example is intended to preliminarily screen for an optimal buffer system and stabilizer to provide optimal stability for the anti-PD-1 antibodies disclosed herein for clinical use.

### 1.1 Procedures

This example was performed with the antibody toripalimab. The sample was concentrated by UF/DF ultrafiltration using a Millipore Pellicon 3 membrane (0.11 m²) to a concentration of about 180 mg/mL, and dialyzed against the corresponding formula shown in Table 3 to a final concentration of about 180 mg/mL, followed by the addition of polysorbate 80 (II) at the corresponding concentration. The solution was aseptically filled into 2R vials at 2.0 mL/vial on a super-clean bench, and stored and tested for stability.

TABLE 3
First round of formula screening - formula information in
preliminary screening for buffer system and stabilizer

| Formula | Buffer | | | Polysorbate | Protein |
|---|---|---|---|---|---|
| No. | system/pH | Stabilizer 1 | Stabilizer 2 | 80 (II) | concentration |
| FS1-1 | 20 mM | 220 mM sucrose | / | 0.02% | 180 mg/mL |
| FS1-2 | histidine | 140 mM arginine | / | | |
| | buffer, | hydrochloride | | | |
| FS1-3 | pH 6.0 | 220 mM trehalose | / | | |
| FS1-4 | | 140 mM sodium | / | | |
| | | chloride | | | |
| FS1-5 | | 130 mM sucrose | 50 mM arginine | | |
| | | | hydrochloride | | |
| FS1-6 | | 130 mM sucrose | 50 mM sodium | | |
| | | | chloride | | |

Note:
“/” indicates none.

### 1.2 Results

### 1.2.1 Appearance and Viscosity Results

According to the results in Table 4, no obvious visible particles and no obvious opalescence were observed in all the formulas at TO. After being subjected to three and five freezing/thawing cycles, all the samples showed no significant change in appearance; after being stored for 1 month under high temperature and long-term conditions, all the samples showed no significant change in appearance; and the formulas FST-1, FST-3 and FST-6 had higher viscosity.

TABLE 4
First round of formula screening - sample appearance data

| | | | High temperature | | | Long-term | FT | | T0 |
|---|---|---|---|---|---|---|---|---|---|
| | Formula | T0 | (40 ± 2° C.) | | | (5 ± 3° C.) | (−40° C./RT) | | Viscosity |
| Test item | No. | Appearance | 1 W | 2 W | 1 M | 1 M | 3 C | 5 C | (cP) |
| Appearance | FS1-1 | CL, | CL, | CL, | CL, | CL, | CL, | CL, | 9.518 |
| | | SO, | SO, | SO, | SO, | SO, | SO, | SO, | |
| | | FVP | FVP | FVP | FVP | FVP | FVP | FVP | |
| | FS1-2 | CL, | CL, | CL, | CL, | CL, | CL, | CL, | 5.531 |
| | | SO, | SO, | SO, | SO, | SO, 1 | SO, | SO, | |
| | | FVP | FVP | FVP | FVP | particle | FVP | FVP | |
| | FS1-3 | CL, | CL, | CL, | CL, | CL, | CL, | CL, | 10.36 |
| | | SO, | SO, | SO, | SO, | SO, | SO, | SO, | |
| | | FVP | FVP | FVP | FVP | FVP | FVP | FVP | |
| | FS1-4 | CL, | CL, | CL, | CL, | CL, | CL, | CL, | 6.288 |
| | | SO, | SO, | SO, | SO, | SO, | SO, | SO, | |
| | | FVP | FVP | FVP | FVP | FVP | FVP | FVP | |
| | FS1-5 | CL, | CL, | CL, | CL, | CL, | CL, | CL, | 6.950 |
| | | SO, | SO, | SO, | SO, | SO, | SO, | SO, | |
| | | FVP | FVP | FVP | FVP | FVP | FVP | FVP | |
| | FS1-6 | CL, | CL, | CL, | CL, | CL, | CL, | CL, | 9.518 |
| | | SO, | SO, | SO, | SO, | SO, | SO, | SO, | |
| | | FVP | FVP | FVP | FVP | FVP | FVP | FVP | |

Note:
“CL” indicates colorless, “SO” indicates slight opalescence, “FVP” indicates free of visible particles, and “1 particle” indicates one particle.

### 1.2.2 Purity Results by SEC

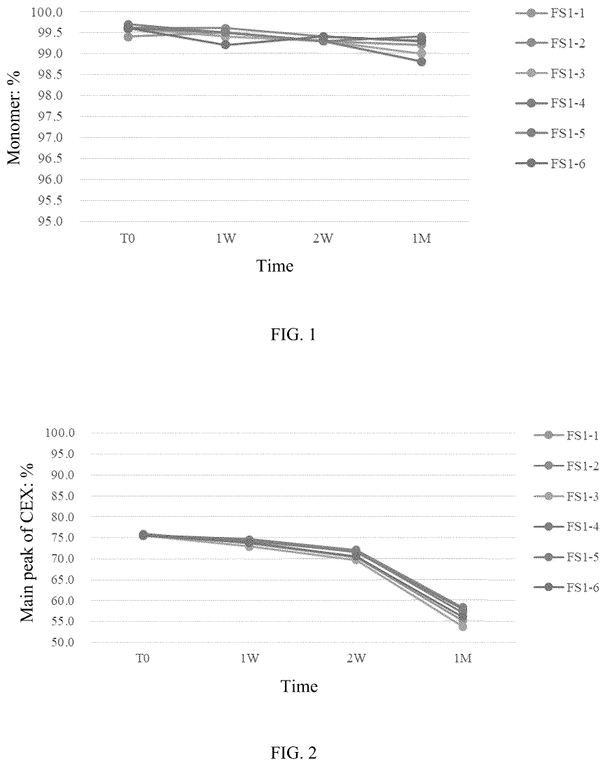

According to the trend of change in purity by SEC-HPLC in FIG. 1 and the purity results by SEC-HPLC in Table 5, after all the formulas were stored for 1 month (1M) under a high temperature condition, formula FS1-4 showed a significant decrease in purity, and all the polymers were significantly increased, while the remaining formulas showed no significant change in purity. After being stored for TM under a long-term condition and subjected to 5 repeated freeze/thawing cycles, all the formulas showed no significant change in the purity by SEC.

TABLE 5
First round of formula screening - SEC-HPLC data

| | | | | | | Long-term | |
|---|---|---|---|---|---|---|---|
| | | | High temperature | | | (5 ± | FT |
| | | | (40 ± 2° C.) | | | 3° C.) | (−40° C./ |
| Formula | SEC- | T0 | (%) | | | (%) | RT) (%) |
| No. | HPLC | (%) | 1 W | 2 W | 1 M | 1 M | 5 C |
| FS1-1 | Poly- | 0.6 | 0.4 | 0.5 | 0.7 | 0.3 | 0.4 |
| | mer % | | | | | | |
| | Mono- | 99.4 | 99.5 | 99.3 | 99.2 | 99.7 | 99.6 |
| | mer % | | | | | | |
| | Frag- | 0.0 | 0.0 | 0.2 | 0.1 | 0.0 | 0.0 |
| | ment % | | | | | | |
| FS1-2 | Poly- | 0.4 | 0.4 | 0.4 | 0.6 | 0.5 | 0.4 |
| | mer % | | | | | | |
| | Mono- | 99.6 | 99.6 | 99.4 | 99.3 | 99.5 | 99.6 |
| | mer % | | | | | | |
| | Frag- | 0.0 | 0.0 | 0.2 | 0.1 | 0.0 | 0.0 |
| | ment % | | | | | | |
| FS1-3 | Poly- | 0.4 | 0.5 | 0.5 | 0.9 | 0.2 | 0.3 |
| | mer % | | | | | | |
| | Mono- | 99.6 | 99.4 | 99.3 | 99.0 | 99.8 | 99.7 |
| | mer % | | | | | | |
| | Frag- | 0.0 | 0.0 | 0.2 | 0.1 | 0.0 | 0.0 |
| | ment % | | | | | | |
| FS1-4 | Poly- | 0.4 | 0.5 | 0.5 | 1.1 | 0.3 | 0.4 |
| | mer % | | | | | | |
| | Mono- | 99.6 | 99.5 | 99.3 | 98.8 | 99.7 | 99.6 |
| | mer % | | | | | | |
| | Frag- | 0.0 | 0.0 | 0.2 | 0.1 | 0.0 | 0.0 |
| | ment % | | | | | | |
| FS1-5 | Poly- | 0.3 | 0.4 | 0.5 | 0.5 | 0.4 | 0.4 |
| | mer % | | | | | | |
| | Mono- | 99.7 | 99.5 | 99.3 | 99.4 | 99.6 | 99.6 |
| | mer % | | | | | | |
| | Frag- | 0.0 | 0.0 | 0.2 | 0.1 | 0.0 | 0.0 |
| | ment % | | | | | | |
| FS1-6 | Poly- | 0.4 | 0.7 | 0.4 | 0.5 | 0.3 | 0.5 |
| | mer % | | | | | | |
| | Mono- | 99.6 | 99.2 | 99.4 | 99.3 | 99.7 | 99.5 |
| | mer % | | | | | | |
| | Frag- | 0.0 | 0.0 | 0.2 | 0.1 | 0.0 | 0.0 |
| | ment % | | | | | | |

### 1.2.3 Purity Results by R-CE-SDS

According to the purity results by R-CE-SDS in Table 6, all the samples showed a decrease in purity by R-CE-SDS after being stored for 1M under a high temperature condition. After being stored for 1M under a long-term condition and subjected to five freeze/thawing cycles, all the sample showed no significant change in purity by R-CE-SDS.

TABLE 6
First round of formula screening - R-CE-SDS data

| | | | | | Long-term | |
|---|---|---|---|---|---|---|
| | | High temperature | | | (5 ± | FT |
| | | (40 ± 2° C.) | | | 3° C.) | (−40° C./ |
| Formula | T0 | (%) | | | (%) | RT) (%) |
| No. | (%) | 1 W | 2 W | 1 M | 1 M | 5 C |
| FS1-1 | 99.5 | 99.4 | 99.1 | 98.1 | 99.5 | 99.6 |
| FS1-2 | 99.5 | 99.4 | 99.0 | 98.2 | 99.5 | 99.4 |
| FS1-3 | 99.5 | 99.4 | 99.0 | 98.5 | 99.4 | 99.6 |
| FS1-4 | 99.4 | 99.4 | 98.8 | 98.3 | 99.6 | 99.6 |
| FS1-5 | 99.5 | 99.3 | 99.0 | 98.4 | 99.5 | 99.6 |
| FS1-6 | 99.5 | 99.2 | 99.2 | 98.4 | 99.5 | 99.6 |

### 1.2.4 Purity Results by NR-CE-SDS

According to the purity results by NR-CE-SDS in Table 7, all the samples showed a significant decrease in purity by NR-CE-SDS after being stored for 1M under a high temperature condition, with the purity of the formulas FS1-4 and FS1-5 decreased relatively rapidly; and all the samples showed no significant change in purity by NR-CE-SDS after being stored for TM under a long-term condition and subjected to five freeze/thawing cycles.

TABLE 7
First round of formula screening - NR-CE-SDS data

| | | | | | Long-term | |
|---|---|---|---|---|---|---|
| | | High temperature | | | (5 ± | FT |
| | | (40 ± 2° C.) | | | 3° C.) | (−40° C./ |
| Formula | T0 | (%) | | | (%) | RT) (%) |
| No. | (%) | 1 W | 2 W | 1 M | 1 M | 5 C |
| FS1-1 | 97.1 | 96.5 | 95.6 | 95.2 | 96.3 | 97.0 |
| FS1-2 | 97.1 | 96.7 | 95.9 | 95.3 | 96.3 | 96.9 |
| FS1-3 | 97.1 | 96.4 | 95.7 | 95.2 | 96.2 | 96.8 |
| FS1-4 | 97.1 | 96.6 | 95.8 | 93.9 | 96.1 | 96.7 |
| FS1-5 | 97.0 | 96.7 | 95.9 | 94.2 | 95.8 | 97.0 |
| FS1-6 | 97.1 | 96.6 | 95.5 | 95.1 | 95.7 | 96.9 |

### 1.2.5 Purity Results by CEX-HPLC

According to the trend of change in purity by CEX-HPLC in FIG. 2 and the purity results by CEX-HPLC in Table 8, all the formulas showed a significant decrease in purity and a significant increase in acid peak after being stored for TM under a high temperature condition, with the purity of the formula FST-3 decreased most rapidly; after all the samples were stored for TM under a long-term condition and subjected to five freeze/thawing cycles, all the formulas showed no significant decrease in purity by CEX.

TABLE 8
First round of formula screening - CEX-HPLC data

| | | | | | | Long- | |
|---|---|---|---|---|---|---|---|
| | | | | | | term | FT |
| | | | High temperature | | | (5 ± | (−40° C./ |
| Formula | | | (40 ± 2° C.) | | | 3° C.) | RT) |
| No. | CEX-HPLC | T0 | 1 W | 2 W | 1 M | 1 M | 5 C |
| FS1-1 | Acid peak % | 16.8 | 21.7 | 24.3 | 41.6 | 16.3 | 15.6 |
| | Main peak % | 75.9 | 73.5 | 70.3 | 55.2 | 76.9 | 78.4 |
| | Basic | 7.3 | 4.8 | 5.4 | 3.2 | 6.8 | 6.0 |
| | peak % | | | | | | |
| FS1-2 | Acid peak % | 16.9 | 20.5 | 23.0 | 38.3 | 15.9 | 15.5 |
| | Main peak % | 75.6 | 74.5 | 72.0 | 58.3 | 77.3 | 79.5 |
| | Basic | 7.5 | 5.0 | 5.0 | 3.3 | 6.8 | 5.0 |
| | peak % | | | | | | |
| FS1-3 | Acid peak % | 17.1 | 22.3 | 25.8 | 42.6 | 16.2 | 15.7 |
| | Main peak % | 75.5 | 72.8 | 69.6 | 53.7 | 77.1 | 78.4 |
| | Basic | 7.4 | 5.0 | 4.6 | 3.6 | 6.7 | 5.9 |
| | peak % | | | | | | |
| FS1-4 | Acid peak % | 17.1 | 21.3 | 23.8 | 39.5 | 16.0 | 15.6 |
| | Main peak % | 75.4 | 74.4 | 71.4 | 57.9 | 77.4 | 78.6 |
| | Basic | 7.4 | 4.3 | 4.7 | 2.6 | 6.6 | 5.8 |
| | peak % | | | | | | |
| FS1-5 | Acid peak % | 17.1 | 21.1 | 23.6 | 39.5 | 15.2 | 15.4 |
| | Main peak % | 75.4 | 74.0 | 71.6 | 57.1 | 78.0 | 78.8 |
| | Basic | 7.5 | 4.4 | 4.8 | 3.4 | 6.7 | 5.8 |
| | peak % | | | | | | |
| FS1-6 | Acid peak % | 17.1 | 21.3 | 24.5 | 40.6 | 16.0 | 15.5 |
| | Main peak % | 75.5 | 73.7 | 70.5 | 56.0 | 77.6 | 78.8 |
| | Basic | 7.4 | 5.0 | 5.0 | 3.4 | 6.3 | 5.7 |
| | peak % | | | | | | |

### 1.2.6 Binding Activity Results

According to the binding activity results in Table 9, all the samples showed no significant change in binding activity after being stored for 1M under a high temperature or long-term condition and subjected to five freeze/thawing cycles.

TABLE 9
First round of formula screening - binding activity data

| | | High temperature | Long-term | FT |
|---|---|---|---|---|
| | | (40 ± 2° C.) | (5 ± 3° C.) | (−40° C./RT) |
| Formula | T0 | (%) | (%) | (%) |
| No. | (%) | 1 M | 1 M | 5 C |
| FS1-1 | 83 | 97 | 103 | 98 |
| FS1-2 | 87 | 106 | 97 | 92 |
| FS1-3 | 87 | 101 | 97 | 103 |
| FS1-4 | 94 | 98 | 101 | 104 |
| FS1-5 | 94 | 108 | 103 | 104 |
| FS1-6 | 105 | 100 | 106 | 99 |

### 1.2.7 Cell Activity Results

According to the cell activity results in Table 10, all the samples showed significant change in cell activity after being stored for 1M under a high temperature or long-term condition and subjected to five freeze/thawing cycles.

TABLE 10
First round of formula screening - cell activity data

| | | High temperature | Long-term | FT |
|---|---|---|---|---|
| | | (40 ± 2° C.) | (5 ± 3° C.) | (−40° C./RT) |
| Formula | T0 | (%) | (%) | (%) |
| No. | (%) | 1 M | 1 M | 5 C |
| FS1-1 | 83 | 111 | 110 | 72 |
| FS1-2 | 87 | 93 | 119 | 68 |
| FS1-3 | 87 | 113 | 113 | 68 |
| FS1-4 | 94 | 129 | 108 | 76 |
| FS1-5 | 94 | 104 | 115 | 84 |
| FS1-6 | 105 | 87 | 107 | 63 |

### 1.2.8 Sub-Visible Particle Results

According to the detection results of sub-visible particles in Table 11, all the formulas showed no significant increase in sub-visible particles after being stored for 1M under a high temperature condition; and all the formulas showed no significant change in sub-visible particles after being stored for 1M under a long-term condition and subjected to 5 repeated freeze/thawing cycles.

TABLE 11
First round of formula screening - sub-visible particle data

| | Accelerated | | Long-term | | | |
|---|---|---|---|---|---|---|
| | (40 ± 2° C.) | | (5 ± 3° C.) | | FT(−40° C./ | |
| | 1 M | | 1 M | | RT) 5 C | |
| Formula | 10 ≤ X < | 25 < | 10 ≤ X < | 25 < | 10 ≤ X < | 25 < |
| No. | 25 μm | X μm | 25 μm | X μm | 25 μm | X μm |
| FS1-1 | 40 | 4 | 167 | 23 | 29 | 18 |
| FS1-2 | 86 | 12 | 35 | 12 | 48 | 18 |
| FS1-3 | 80 | 4 | 52 | 6 | 44 | 14 |
| FS1-4 | 113 | 11 | 36 | 2 | 31 | 12 |
| FS1-5 | 258 | 29 | 52 | 8 | 27 | 12 |
| FS1-6 | 167 | 12 | 76 | 7 | 52 | 26 |

### 1.3 Conclusion of the First Round of Formula Screening

According to the appearance results, no significant differences are observed among the formulas. According to the viscosity results, the formulas FS1-1, FS1-3 and FS1-6 have higher viscosity. According to the SEC-HPLC results, the formula FS1-4 results in a higher number of polymers. According to the purity results by NR-CE-SDS, the formulas FS1-4 and FS1-5 have a lower purity. According to the purity results by CEX-HPLC, the purity by CEX of the formula FS1-3 decreases relatively rapidly. According to the purity results by R-CE-SDS, the binding activity results and the cell activity results, all the formulas show no difference; and according to the sub-visible particle results, the formula FS1-4 results in a higher number of sub-visible particles, and there is no significant difference among other formulas.

In conclusion, the formulas FS1-2 and FS1-5 have superior overall performances, and sucrose and arginine hydrochloride are selected as stabilizers to enter the next round of screening.

### Example 2. Screening for pH and Excipient and Comparison Test on Low-Concentration Formulas

In order to further explore the effect of different excipients on the stability of the antibody, one of sodium chloride, sucrose, arginine hydrochloride, glycine and mannitol was selected for a comparison test. The effect of the different excipients on the stability of the antibody toripalimab at a concentration of 180 mg/mL was investigated in a 20 mM histidine buffer system at pH 6.0, a 20 mM histidine buffer system at pH 5.5, and a 20 mM citric acid buffer system at pH 6.0.

### 2.1 Procedures

This example was performed with the antibody toripalimab. The sample was concentrated by UF/DF ultrafiltration using a Millipore Pellicon 12 membrane (0.11 m²) to a concentration of about 180 mg/mL, and dialyzed against the corresponding formula shown in Table 3 to a final concentration of about 180 mg/mL, followed by the addition of polysorbate 80 (II) at the corresponding concentration. The solution was aseptically filled into 2R vials at 2.0 mL/vial on a super-clean bench, and stored and tested for stability.

TABLE 12
Second round of formula screening - formula information in screening for
pH and excipient and comparison test on low-concentration formulas

| Formula | Buffer | | | | Protein |
|---|---|---|---|---|---|
| No. | system/pH | Stabilizer 1 | Stabilizer 2 | Surfactant | concentration |
| FS2-1 | 20 mM | 140 mM sucrose | 50 mM arginine | 0.02% | 180 mg/mL |
| | histidine | | hydrochloride | polysorbate | |
| FS2-2 | buffer, | / | 140 mM arginine | 80 (II) | |
| | pH 6.0 | | hydrochloride | | |
| FS2-3 | | 100 mM glycine | 50 mM arginine | | |
| | | | hydrochloride | | |
| FS2-4 | 20 mM | 150 mM mannitol | 50 mM sodium | | |
| | citric acid | | chloride | | |
| | buffer, | | | | |
| | pH 6.0 | | | | |
| FS2-5 | 20 mM | 140 mM sucrose | 50 mM arginine | | |
| | histidine | | hydrochloride | | |
| FS2-6 | buffer, | / | 140 mM arginine | | |
| | pH 5.5 | | hydrochloride | | |
| FS2-7 | | 100 mM glycine | 50 mM arginine | | |
| | | | hydrochloride | | |

Note:
“/” indicates none.

### 2.2 Results

### 2.2.1 Appearance and Concentration Results

According to the results in Table 13, all the samples showed no significant change in protein content after being stored for 1M under high temperature and long-term conditions; no obvious visible particles and no obvious opalescence were observed in all the samples after being stored for 1M under a high temperature, long-term or accelerated condition; and the formulas FS2-2 and FS2-6 showed superior viscosity results.

TABLE 13
Second round of formula screening - protein content data

| | | | | | | | | Long-term | FT | |
|---|---|---|---|---|---|---|---|---|---|---|
| | | | High temperature | | | Accelerated | | (5 ± | (−40° C./ | |
| | Formula | | (40 ± 2° C.) | | | (25 ± 2° C.) | | 3° C.) | RT) | |
| Test item | No. | T0 | 1 W | 2 W | 1 M | 2 W | 1 M | 1 M | 3 C | 5 C |
| Protein | FS2-1 | 172.6 | / | / | 173.7 | / | 177.7 | 173.3 | / | / |
| concentration | FS2-2 | 175.0 | / | / | 172.7 | / | 168.8 | 173.9 | / | / |
| (mg/mL) | FS2-3 | 176.3 | / | / | 174.3 | / | 169.2 | 180.5 | / | / |
| | FS2-4 | 177.7 | / | / | 175.1 | / | 173.5 | 171.2 | / | / |
| | FS2-5 | 175.2 | / | / | 172.5 | / | 176.1 | 176.9 | / | / |
| | FS2-6 | 173.8 | / | / | 174.9 | / | 175.3 | 174.7 | / | / |
| | FS2-7 | 176.4 | / | / | 179.7 | / | 181.0 | 176.4 | / | / |
| Appearance | FS2-1 | CL, SO, | CL, SO, | CL, SO, | CL, SO, | CL, SO, | CL, SO, | CL, SO, | CL, SO, | CL, SO, |
| | | FVP | FVP | FVP | FVP | FVP | FVP | FVP | FVP | FVP |
| | FS2-2 | CL, SO, | CL, SO, | CL, SO, | CL, SO, | CL, SO, | CL, SO, | CL, SO, | CL, SO, | CL, SO, |
| | | 1 fiber | FVP | 1 fiber | FVP | FVP | FVP | FVP | 1 fiber | FVP |
| | FS2-3 | CL, SO, | CL, SO, | CL, SO, | CL, SO, | CL, SO, | CL, SO, | CL, SO, | CL, SO, | CL, SO, |
| | | 1 fiber | FVP | FVP | FVP | 1 fiber | 1 fiber | FVP | FVP | FVP |
| | FS2-4 | CL, SO, | CL, SO, | CL, SO, | CL, SO, | CL, SO, | CL, SO, | CL, SO, | CL, SO, | CL, SO, |
| | | FVP | FVP | FVP | FVP | FVP | FVP | FVP | FVP | FVP |
| | FS2-5 | CL, SO, | CL, SO, | CL, SO, | CL, SO, | CL, SO, | CL, SO, | CL, SO, | CL, SO, | CL, SO, |
| | | 1 fiber | FVP | FVP | FVP | FVP | FVP | FVP | 1 fiber | FVP |
| | FS2-6 | CL, SO, | CL, SO, | CL, SO, | CL, SO, | CL, SO, | CL, SO, | CL, SO, | CL, SO, | CL, SO, |
| | | FVP | FVP | FVP | FVP | FVP | FVP | FVP | FVP | FVP |
| | FS2-7 | CL, SO, | CL, SO, | CL, SO, | CL, SO, | CL, SO, | CL, SO, | CL, SO, | CL, SO, | CL, SO, |
| | | FVP | FVP | FVP | 1 fiber | FVP | FVP | FVP | FVP | FVP |
| Viscosity | FS2-1 | 8.399 | / | / | / | / | / | / | / | / |
| (cP) | FS2-2 | 7.134 | / | / | / | / | / | / | / | / |
| | FS2-3 | 8.069 | / | / | / | / | / | / | / | / |
| | FS2-4 | 8.214 | / | / | / | / | / | / | / | / |
| | FS2-5 | 8.809 | / | / | / | / | / | / | / | / |
| | FS2-6 | 7.090 | / | / | / | / | / | / | / | / |
| | FS2-7 | 8.027 | / | / | / | / | / | / | / | / |

Note:
“CL” indicates colorless, “SO” indicates slight opalescence, “FVP” indicates free of visible particles, “1 fiber” indicates one fiber, and “/” indicates not detected.

### 2.2.2 Purity Results by SEC-HPLC

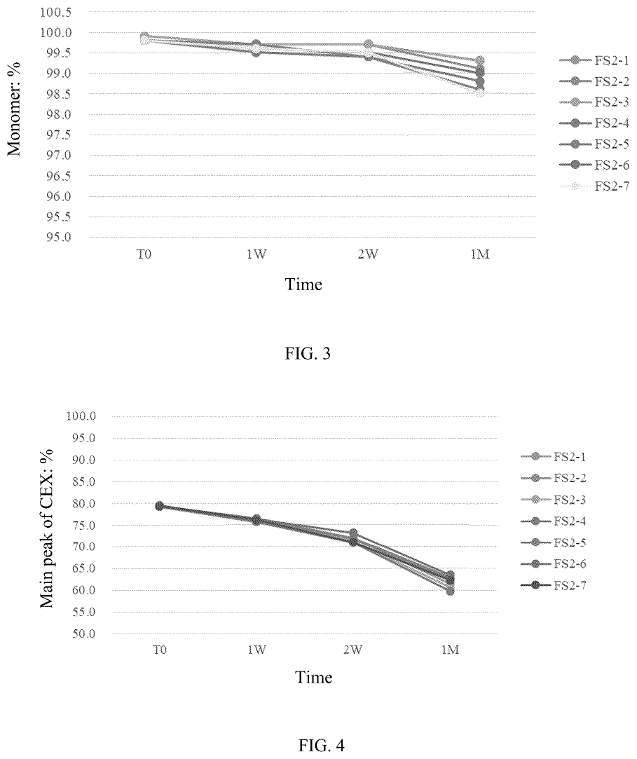

According to the trend of change in purity by SEC-HPLC in FIG. 3 and the purity results by SEC-HPLC in Table 14, all the samples showed a decrease in purity by SEC after being stored for 1M under a high temperature condition, with the purity by SEC of the formulas FS2-4, FS2-5 and FS2-7 decreased relatively rapidly; and all the samples showed no significant change in purity by SEC after being stored for 1M under an accelerated or long-term condition.

TABLE 14
Second round of formula screening - purity data by SEC-HPLC

| | | | High temperature | | | Accelerated | | Long-term | FT | |
|---|---|---|---|---|---|---|---|---|---|---|
| | | | (40 ± 2° C.) | | | (25 ± 2° C.) | | (5 ± 3° C.) | (−40° C./ | |
| Formula | SEC- | T0 | (%) | | | (%) | | (%) | RT) (%) | |
| No. | HPLC | (%) | 1 W | 2 W | 1 M | 2 W | 1 M | 1 M | 3 C | 5 C |
| FS2-1 | Polymer % | 0.1 | 0.2 | 0.2 | 0.3 | 0.2 | 0.2 | 0.2 | 0.2 | 0.1 |
| | Monomer % | 99.9 | 99.7 | 99.7 | 99.3 | 99.8 | 99.8 | 99.8 | 99.8 | 99.9 |
| | Fragment % | 0.0 | 0.1 | 0.1 | 0.3 | 0.0 | 0.0 | 0.0 | 0.0 | 0.0 |
| FS2-2 | Polymer % | 0.2 | 0.3 | 0.2 | 0.5 | 0.2 | 0.2 | 0.3 | 0.1 | 0.2 |
| | Monomer % | 99.8 | 99.7 | 99.7 | 99.1 | 99.8 | 99.7 | 99.7 | 99.9 | 99.8 |
| | Fragment % | 0.0 | 0.1 | 0.1 | 0.3 | 0.0 | 0.0 | 0.0 | 0.0 | 0.0 |
| FS2-3 | Polymer % | 0.1 | 0.2 | 0.2 | 0.3 | 0.1 | 0.3 | 0.2 | 0.1 | 0.1 |
| | Monomer % | 99.9 | 99.7 | 99.7 | 99.3 | 99.9 | 99.6 | 99.8 | 99.9 | 99.9 |
| | Fragment % | 0.0 | 0.1 | 0.1 | 0.4 | 0.0 | 0.0 | 0.0 | 0.0 | 0.0 |
| FS2-4 | Polymer % | 0.2 | 0.4 | 0.4 | 0.8 | 0.3 | 0.7 | 0.3 | 0.2 | 0.3 |
| | Monomer % | 99.8 | 99.5 | 99.4 | 98.8 | 99.7 | 99.3 | 99.7 | 99.8 | 99.7 |
| | Fragment % | 0.0 | 0.1 | 0.1 | 0.3 | 0.0 | 0.0 | 0.0 | 0.0 | 0.0 |
| FS2-5 | Polymer % | 0.2 | 0.3 | 0.5 | 1.0 | 0.2 | 0.5 | 0.4 | 0.2 | 0.3 |
| | Monomer % | 99.8 | 99.7 | 99.4 | 98.6 | 99.8 | 99.5 | 99.6 | 99.8 | 99.7 |
| | Fragment % | 0.0 | 0.1 | 0.1 | 0.4 | 0.0 | 0.0 | 0.0 | 0.0 | 0.0 |
| FS2-6 | Polymer % | 0.2 | 0.3 | 0.3 | 0.6 | 0.2 | 0.5 | 0.3 | 0.2 | 0.2 |
| | Monomer % | 99.8 | 99.6 | 99.5 | 99.0 | 99.8 | 99.5 | 99.7 | 99.8 | 99.8 |
| | Fragment % | 0.0 | 0.1 | 0.1 | 0.4 | 0.0 | 0.0 | 0.0 | 0.0 | 0.0 |
| FS2-7 | Polymer % | 0.2 | 0.3 | 0.4 | 1.1 | 0.2 | 0.4 | 0.3 | 0.2 | 0.3 |
| | Monomer % | 99.8 | 99.6 | 99.5 | 98.5 | 99.8 | 99.5 | 99.7 | 99.8 | 99.7 |
| | Fragment % | 0.0 | 0.1 | 0.1 | 0.4 | 0.0 | 0.0 | 0.0 | 0.0 | 0.0 |

### 2.2.3 Purity Results by R-CE-SDS

According to the purity results by R-CE-SDS in Table 15, all the samples showed a decreased in the purity after being stored for 1M under a high temperature (40° C.) condition; and all the samples showed no significant change in the purity after being stored for 1M under an accelerated (25° C.) condition or long-term condition. There was no inter-group difference in the samples.

TABLE 15
Second round of formula screening - purity data by R-CE-SDS

| | | High temperature | | | Accelerated | | Long-term | FT | |
|---|---|---|---|---|---|---|---|---|---|
| | | (40 ± 2° C.) | | | (25 ± 2° C.) | | (5 ± 3° C.) | (−40° C./ | |
| Formula | T0 | (%) | | | (%) | | (%) | RT) (%) | |
| No. | (%) | 1 W | 2 W | 1 M | 2 W | 1 M | 1 M | 3 C | 5 C |
| FS2-1 | 99.5 | 99.4 | 99.0 | 98.6 | 99.2 | 99.4 | 99.4 | 99.6 | 99.7 |
| FS2-2 | 99.4 | 99.3 | 98.9 | 98.5 | 99.3 | 99.3 | 99.3 | 99.6 | 99.2 |
| FS2-3 | 99.6 | 99.4 | 98.7 | 98.5 | 99.2 | 99.4 | 99.3 | 99.6 | 99.6 |
| FS2-4 | 99.5 | 99.5 | 98.8 | 98.5 | 99.1 | 99.4 | 99.2 | 99.7 | 99.6 |
| FS2-5 | 99.5 | 99.3 | 98.9 | 98.7 | 99.2 | 99.4 | 99.2 | 99.6 | 99.6 |
| FS2-6 | 99.4 | 99.3 | 98.9 | 98.6 | 99.2 | 99.3 | 99.0 | 99.6 | 99.6 |
| FS2-7 | 99.6 | 99.3 | 98.8 | 98.6 | 99.3 | 99.3 | 99.2 | 99.7 | 99.6 |

### 2.2.4 Purity Results by NR-CE-SDS

According to the purity results by NR-CE-SDS in Table 16, all the samples showed no significant change in purity after being stored for 1M under a high temperature (40° C.) condition, an accelerated (25° C.) condition or a long-term condition.

TABLE 16
Second round of formula screening - purity data by NR-CE-SDS

| | | High temperature | | | Accelerated | | Long-term | FT | |
|---|---|---|---|---|---|---|---|---|---|
| | | (40 ± 2° C.) | | | (25 ± 2° C.) | | (5 ± 3° C.) | (−40° C./ | |
| Formula | T0 | (%) | | | (%) | | (%) | RT) (%) | |
| No. | (%) | 1 W | 2 W | 1 M | 2 W | 1 M | 1 M | 3 C | 5 C |
| FS2-1 | 96.8 | 96.5 | 96.2 | 95.9 | 95.9 | 96.6 | 96.9 | 97.0 | 96.8 |
| FS2-2 | 97.0 | 96.5 | 96.1 | 95.9 | 95.9 | 96.6 | 96.8 | 96.7 | 96.7 |
| FS2-3 | 97.0 | 96.4 | 96.1 | 96.0 | 95.6 | 96.6 | 96.6 | 96.7 | 96.8 |
| FS2-4 | 97.0 | 96.3 | 95.9 | 95.8 | 95.8 | 96.6 | 96.5 | 96.8 | 96.4 |
| FS2-5 | 96.9 | 96.4 | 96.2 | 96.1 | 95.7 | 96.7 | 96.6 | 96.7 | 96.6 |
| FS2-6 | 96.9 | 96.4 | 95.9 | 96.0 | 95.9 | 96.5 | 96.6 | 96.6 | 96.5 |
| FS2-7 | 96.8 | 96.2 | 96.0 | 95.7 | 95.7 | 96.7 | 96.3 | 96.7 | 96.3 |

### 2.2.5 Purity Results by CEX-HPLC

According to the trend of change in purity by CEX-HPLC in FIG. 4 and the CEX-HPLC results in Table 17, all the samples showed a decrease in purity by CEX-HPLC after being stored for 1M under a high temperature condition, with the purity of the formula FS2-4 decreased relatively rapidly; and all the samples showed no significant change in purity by CEX-HPLC after being stored for 1M under accelerated and long-term conditions.

TABLE 17
Second round of formula screening - purity data by CEX-HPLC

| | | | | | | | | | FT | |
|---|---|---|---|---|---|---|---|---|---|---|
| | | | High temperature | | | Accelerated | | Long-term | (−40° C./ | |
| Formula | | | (40 ± 2° C.) | | | (25 ± 2° C.) | | (5 ± 3° C.) | RT) | |
| No. | CEX-HPLC | T0 | 1 W | 2 W | 1 M | 2 W | 1 M | 1 M | 3 C | 5 C |
| FS2-1 | Acid peak % | 14.1 | 18.9 | 24.1 | 35.9 | 15.4 | 15.9 | 13.9 | 14.5 | 14.6 |
| | Main peak % | 79.5 | 75.8 | 71.9 | 61.4 | 79.0 | 78.7 | 79.7 | 79.0 | 78.9 |
| | Basic peak % | 6.5 | 5.3 | 4.0 | 2.7 | 5.6 | 5.3 | 6.4 | 6.5 | 6.5 |
| FS2-2 | Acid peak % | 14.3 | 18.4 | 23.8 | 34.8 | 15.3 | 15.8 | 14.2 | 14.3 | 14.4 |
| | Main peak % | 79.3 | 76.5 | 72.0 | 63.0 | 79.0 | 79.0 | 79.3 | 79.2 | 79.1 |
| | Basic peak % | 6.5 | 5.1 | 4.2 | 2.2 | 5.7 | 5.2 | 6.4 | 6.4 | 6.5 |
| FS2-3 | Acid peak % | 14.4 | 19.1 | 24.9 | 36.3 | 15.6 | 16.0 | 14.1 | 14.3 | 14.5 |
| | Main peak % | 79.2 | 76.0 | 71.3 | 60.6 | 78.9 | 78.6 | 79.5 | 79.2 | 79.1 |
| | Basic peak % | 6.4 | 4.9 | 3.8 | 3.1 | 5.5 | 5.5 | 6.4 | 6.5 | 6.4 |
| FS2-4 | Acid peak % | 14.3 | 19.3 | 26.1 | 37.1 | 15.6 | 16.0 | 14.3 | 14.5 | 14.5 |
| | Main peak % | 79.2 | 75.7 | 70.9 | 59.7 | 78.8 | 78.6 | 79.2 | 79.1 | 79.1 |
| | Basic peak % | 6.4 | 5.0 | 3.0 | 3.1 | 5.6 | 5.4 | 6.5 | 6.5 | 6.4 |
| FS2-5 | Acid peak % | 14.3 | 17.5 | 22.9 | 32.6 | 15.5 | 15.1 | 14.6 | 14.5 | 14.6 |
| | Main peak % | 79.2 | 76.3 | 71.8 | 62.5 | 78.5 | 78.7 | 78.9 | 79.0 | 79.0 |
| | Basic peak % | 6.5 | 6.2 | 5.2 | 4.9 | 6.0 | 6.2 | 6.6 | 6.5 | 6.5 |
| FS2-6 | Acid peak % | 14.3 | 17.6 | 22.0 | 31.6 | 15.3 | 14.9 | 14.0 | 14.3 | 14.6 |
| | Main peak % | 79.2 | 76.3 | 73.2 | 63.5 | 78.8 | 79.0 | 79.5 | 79.2 | 79.0 |
| | Basic peak % | 6.5 | 6.1 | 4.8 | 4.9 | 6.0 | 6.1 | 6.5 | 6.5 | 6.5 |
| FS2-7 | Acid peak % | 14.3 | 18.2 | 23.8 | 33.2 | 15.2 | 15.5 | 13.5 | 14.3 | 14.4 |
| | Main peak % | 79.3 | 76.1 | 71.0 | 62.2 | 78.8 | 78.3 | 80.1 | 79.2 | 79.1 |
| | Basic peak % | 6.5 | 5.7 | 5.2 | 4.6 | 5.9 | 6.2 | 6.4 | 6.5 | 6.4 |

### 2.2.6 Cell Activity Results

According to the cell activity results in Table 18, all the samples showed no significant change in cell activity after being stored for 1M under high temperature, accelerated and long-term conditions and subjected to three and five freeze/thawing cycles.

TABLE 18
Second round of formula screening - cell activity data

| | | High temperature | | | Accelerated | Long-term | FT | |
|---|---|---|---|---|---|---|---|---|
| | | (40 ± 2° C.) | | | (25 ± 2° C.) | (5 ± 3° C.) | (−40° C./ | |
| Formula | T0 | (%) | | | (%) | (%) | RT) (%) | |
| No. | (%) | 1 W | 2 W | 1 M | 1 M | 1 M | 3 C | 5 C |
| FS2-1 | 109 | 94 | 106 | 114 | 101 | 95 | 109 | 94 |
| FS2-2 | 95 | 95 | 112 | 111 | 106 | 88 | 95 | 95 |
| FS2-3 | 103 | 100 | 91 | 114 | 104 | 98 | 103 | 100 |
| FS2-4 | 113 | 91 | 96 | 123 | 96 | 90 | 113 | 91 |
| FS2-5 | 116 | 97 | 86 | 110 | 100 | 109 | 116 | 97 |
| FS2-6 | 116 | 80 | 112 | 92 | 99 | 94 | 116 | 80 |
| FS2-7 | 79 | 106 | 114 | 114 | 108 | 107 | 79 | 106 |

### 2.2.7 Binding Activity Results

According to the binding activity results in Table 19, all the samples showed no significant change in binding activity after being stored for TM under high temperature, accelerated and long-term conditions and subjected to three and five freeze/thawing cycles.

TABLE 19
Second round of formula screening - binding activity data

| | | | | | Accel- | Long- | | |
|---|---|---|---|---|---|---|---|---|
| | | | | | erated | term | | |
| | | High temperature | | | (25 ± | (5 ± | FT | |
| | | (40 ± 2° C.) | | | 2° C.) | 3° C.) | (−40° C./ | |
| Formula | T0 | (%) | | | (%) | (%) | RT) (%) | |
| No. | (%) | 1 W | 2 W | 1 M | 1 M | 1 M | 3 C | 5 C |
| FS2-1 | 109 | 100 | 101 | 106 | 98 | 98 | 101 | 104 |
| FS2-2 | 106 | 101 | 95 | 104 | 95 | 96 | 100 | 115 |
| FS2-3 | 102 | 99 | 104 | 100 | 91 | 98 | 103 | 108 |
| FS2-4 | 107 | 99 | 108 | 106 | 94 | 94 | 109 | 109 |
| FS2-5 | 102 | 97 | 109 | 105 | 93 | 95 | 103 | 111 |
| FS2-6 | 105 | 101 | 96 | 98 | 100 | 97 | 121 | 108 |
| FS2-7 | 99 | 106 | 102 | 101 | 99 | 96 | 113 | 108 |

### 2.2.8 Sub-Visible Particle Results

According to the sub-visible particle results in Table 20, all the samples showed no significant change in sub-visible particles after being stored for TM under an accelerated or long-term condition.

TABLE 20
Second round of formula screening - sub-visible particle data

| | | | | | | | Accelerated | | Long-term | | FT | | | |
|---|---|---|---|---|---|---|---|---|---|---|---|---|---|---|
| | High temperature | | | | | | (25 ± | | (5 ± | | (−40° C./ | | | |
| | (40 ± 2° C.) | | | | | | 2° C.) | | 3° C.) | | RT) | | | |
| | 1 W | | 2 W | | 1 M | | 1 M | | 1 M | | 3 C | | 5 C | |
| | 10 ≤ | | 10 ≤ | | 10 ≤ | | 10 ≤ | | 10 ≤ | | 10 ≤ | | 10 ≤ | |
| Formula | X < | 25 μm < | X < | 25 μm < | X < | 25 μm < | X < | 25 μm < | X < | 25 μm < | X < | 25 μm < | X < | 25 μm < |
| No. | 25 μm | X | 25 μm | X | 25 μm | X | 25 μm | X | 25μm | X | 25 μm | X | 25 μm | X |
| FS2-1 | 26 | 2 | 36 | 4 | 16 | 10 | 48 | 0 | 24 | 2 | 52 | 5 | 27 | 1 |
| FS2-2 | 100 | 7 | 9 | 0 | 199 | 21 | 21 | 2 | 50 | 0 | 70 | 4 | 82 | 10 |
| FS2-3 | 34 | 8 | 19 | 1 | 35 | 6 | 21 | 0 | 40 | 10 | 195 | 0 | 119 | 12 |
| FS2-4 | 22 | 0 | 20 | 2 | 54 | 10 | 50 | 0 | 19 | 8 | 118 | 22 | 22 | 2 |
| FS2-5 | 10 | 2 | 22 | 4 | 33 | 0 | 57 | 8 | 63 | 4 | 41 | 11 | 18 | 3 |
| FS2-6 | 16 | 0 | 14 | 4 | 48 | 4 | 23 | 6 | 91 | 12 | 75 | 6 | 53 | 10 |
| FS2-7 | 78 | 4 | 29 | 1 | 110 | 14 | 57 | 8 | 71 | 14 | 34 | 2 | 96 | 5 |

### 2.3 Conclusion of the Second Round of Formula Screening

According to the protein content, appearance, purity results by R-CE-SDS, purity results by NR-CE-SDS, purity results by CEX-HPLC, cell activity, binding activity results, and sub-visible particle results, no significant differences are observed among the formulas. According to the viscosity results, the formulas FS2-2 and FS2-6 are superior; according to the purity results by SEC-HPLC, the purity of the formulas FS2-4, FS2-5 and FS2-7 decreases relatively rapidly, among which FS2-4 is a low-concentration formula determined by the original intravenous injection (CN application No. 201610628048.6), which is experimentally found not to be suitable for high-concentration antibody formulations.

In conclusion, the formula FS2-2 (20 mM histidine buffer, pH 6.0, including 140 mM arginine hydrochloride) is finally selected. In order to keep the osmotic pressure at about 300 mOsm/kg for better use in subcutaneous injections, the formula is further adjusted to increase the content of arginine hydrochloride to 150 mM, and the concentration of Tween in this formula is further investigated.

### Example 3. Screening for Surfactant

Surfactants are commonly added in liquid formulations as an agent for protecting proteins such as antibodies from air/solution interface-induced stress or solution/surface-induced stress during storage to reduce the aggregation of the antibodies or minimize the formation of particles in the formulation, which facilitates the stability of the physicochemical properties of the antibodies. Polysorbate 80 at different concentrations were added into the formulations including 20 mM histidine buffer and 180 mg/mL of antibody toripalimab to investigate the effect of different concentrations of surfactants described above on stability.

### 3.1 Procedures

This example was performed with the antibody toripalimab. The sample was concentrated by UF/DF ultrafiltration using a Millipore Pellicon 21 membrane (0.11 m²) to a concentration of about 180 mg/mL, and dialyzed against the corresponding formula shown in Table 3 to a final concentration of about 180 mg/mL, followed by the addition of polysorbate 80 (II) at the corresponding concentration. The solution was aseptically filled into 1 mL pre-filled syringes at 1.1 mL/syringe on a super-clean bench, and stored and tested for stability.

TABLE 21
Third round of screening - formula information
in screening for surfactant

| | Protein | | | | |
|---|---|---|---|---|---|
| Formula | concentration | Buffer | | | Polysorbate |
| No. | (mg/mL) | system | pH | Stabilizer | 80 (II) |
| 1 | 180 | 20 mM | 6.0 | 150 mM | 0.02% |
| 2 | | histidine | | arginine | 0.04% |
| 3 | | buffer | | hydrochloride | 0.06% |

### 3.2 Results

### 3.2.1 Appearance and Concentration Results

According to the results in Table 22, all the samples showed no significant change in protein content; and no obvious visible particles and no obvious opalescence were observed from the appearance of all the samples.

TABLE 22
Third round of formula screening - protein content and appearance data

| | | | High temperature | | Long-term |
|---|---|---|---|---|---|
| | Formula | | (40 ± 2° C.) | | (5 ± 3° C.) |
| Test item | No. | T0 | 2 W | 1 M | 1 M |
| Protein | 1 | 173.2 | 176.2 | 177.0 | 173.4 |
| concentration | 2 | 176.0 | 172.8 | 173.1 | 174.1 |
| (mg/mL) | 3 | 170.9 | 168.5 | 170.8 | 174.3 |
| Appearance | 1 | CL, SO, FVP | CL, SO, FVP | CL, SO, FVP | CL, SO, FVP |
| | 2 | CL, SO, FVP | CL, SO, FVP | CL, SO, FVP | CL, SO, FVP |
| | 3 | CL, SO, FVP | CL, SO, FVP | CL, SO, FVP | CL, SO, FVP |

Note:
“CL” indicates colorless,
“SO” indicates slight opalescence, and
“FVP” indicates free of visible particles

### 3.2.2 Purity Results by SEC-HPLC

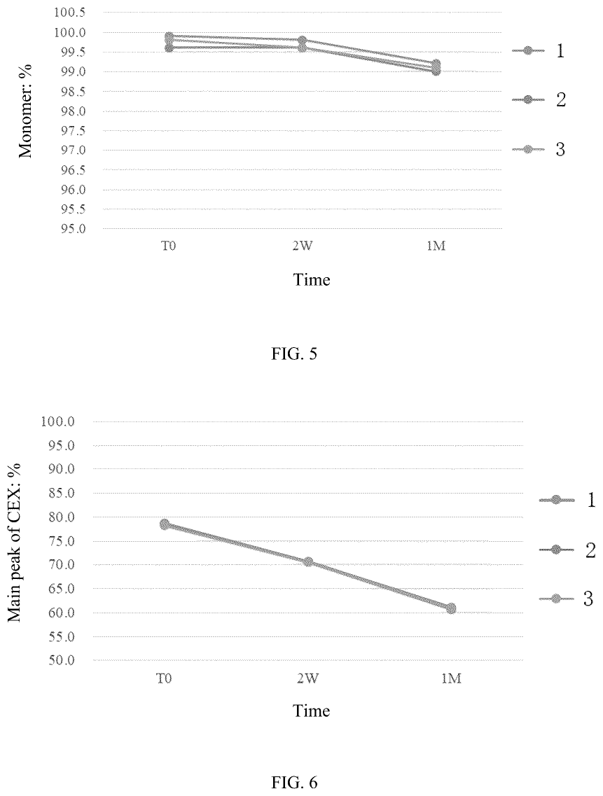

According to the trend of change in purity by SEC-HPLC in FIG. 5 and the purity results by SEC-HPLC in Table 23, all the samples showed no significant change in purity.

TABLE 23
Third round of formula screening - purity data by SEC-HPLC

| | | | High temperature | | Long-term |
|---|---|---|---|---|---|
| Formula | | | (40 ± 2° C.) | | (5 ± 3° C.) |
| No. | SEC-HPLC | T0 | 2 W | 1 M | 1 M |
| 1 | Polymer % | 0.1 | 0.2 | 0.4 | 0.2 |
| | Monomer % | 99.9 | 99.8 | 99.2 | 99.8 |
| | Fragment % | 0.0 | 0.0 | 0.4 | 0.0 |
| 2 | Polymer % | 0.4 | 0.3 | 0.5 | 0.3 |
| | Monomer % | 99.6 | 99.6 | 99.0 | 99.7 |
| | Fragment % | 0.0 | 0.0 | 0.5 | 0.0 |
| 3 | Polymer % | 0.2 | 0.4 | 0.4 | 0.2 |
| | Monomer % | 99.8 | 99.6 | 99.1 | 99.8 |
| | Fragment % | 0.0 | 0.0 | 0.5 | 0.0 |

### 3.2.3 Results by R-CE-SDS

According to the purity results by R-CE-SDS in Table 24, all the samples showed no significant change.

TABLE 24
Third round of formula screening - purity data by R-CE-SDS

| | | | |
|---|---|---|---|
| | | High temperature | Long-term |
| Formula | T0 | (40 ± 2° C.) (%) | (5 ± 3° C.) (%) |

| No. | (%) | 2 W | 1 M | 1 M |
|---|---|---|---|---|
| 1 | 98.9 | 98.9 | 98.5 | 99.5 |
| 2 | 99.2 | 98.8 | 98.4 | 99.6 |
| 3 | 99.3 | 98.9 | 98.1 | 99.6 |

### 3.2.4 Results by NR-CE-SDS

According to the purity results by NR-CE-SDS in Table 25, all the samples showed no significant change.

TABLE 25
Second round of formula screening - purity data by NR-CE-SDS

| | | | |
|---|---|---|---|
| | | High temperature | Long-term |
| Formula | T0 | (40 ± 2° C.) (%) | (5 ± 3° C.) (%) |

| No. | (%) | 2 W | 1 M | 1 M |
|---|---|---|---|---|
| 1 | 97.0 | 96.4 | 96.0 | 97.6 |
| 2 | 97.0 | 96.6 | 96.1 | 97.6 |
| 3 | 97.1 | 96.4 | 96.2 | 97.6 |

### 3.2.5 Purity Results by CEX-HPLC

According to the trend of change in purity by CEX-HPLC in FIG. 6 and the purity results by CEX-HPLC in Table 26, all the samples showed no significant change in purity.

TABLE 26
Third round of formula screening - purity data by CEX-HPLC

| | | | High temperature | | Long-term |
|---|---|---|---|---|---|
| Formula | | | (40 ± 2° C.) | | (5 ± 3° C.) |
| No. | CEX-HPLC | T0 | 2 W | 1 M | 1 M |
| 1 | Acid peak % | 14.9 | 24.6 | 35.8 | 14.6 |
| | Main peak % | 78.5 | 70.6 | 61.0 | 78.9 |
| | Basic peak % | 6.5 | 4.8 | 3.2 | 6.5 |
| 2 | Acid peak % | 15.0 | 24.7 | 35.7 | 14.9 |
| | Main peak % | 78.4 | 70.5 | 60.6 | 78.3 |
| | Basic peak % | 6.6 | 4.8 | 3.7 | 6.8 |
| 3 | Acid peak % | 15.2 | 24.6 | 35.7 | 15.0 |
| | Main peak % | 78.2 | 70.5 | 60.8 | 78.2 |
| | Basic peak % | 6.5 | 4.9 | 3.6 | 6.7 |

### 3.2.6 Binding Activity Results

According to the binding activity results in Table 27, all the samples showed no significant change in activity.

TABLE 27
Third round of formula screening - binding activity data

| | | | |
|---|---|---|---|
| | | High temperature | Long-term |
| Formula | T0 | (40 ± 2° C.) (%) | (5 ± 3° C.) (%) |

| No. | (%) | 2 W | 1 M | 1 M |
|---|---|---|---|---|
| 1 | 105 | 102 | 106 | 107 |
| 2 | 116 | 108 | 107 | 104 |
| 3 | 98 | 99 | 110 | 109 |

### 3.2.7 Cell Activity Results

According to the cell activity results in Table 28, all the samples showed no significant change in activity.

TABLE 28
Third round of formula screening - cell activity data

| | | | |
|---|---|---|---|
| | | High temperature | Long-term |
| Formula | T0 | (40 ± 2° C.) (%) | (5 ± 3° C.) (%) |

| No. | (%) | 2 W | 1 M | 1 M |
|---|---|---|---|---|
| 1 | 107 | 108 | 107 | 121 |
| 2 | 103 | 112 | 119 | 116 |
| 3 | 85 | 108 | 130 | 119 |

### 3.2.8 Sub-Visible Particle Results

According to the sub-visible particle results in Table 29, all the samples showed no significant change.

TABLE 29
Third round of formula screening - sub-visible particle data

| | | | High temperature | | | | Long-term | |
|---|---|---|---|---|---|---|---|---|
| | | | (40 ± 2° C.) | | | | (5 ± 3° C.) | |
| | T0 | | 2 W | | 1 M | | 1 M | |
| Formula | 10 ≤ X < | 25 μm < | 10 ≤ X < | 25 μm < | 10 ≤ X < | 25 μm < | 10 ≤ X < | 25 μm < |
| No. | 25 μm | X | 25 μm | X | 25 μm | X | 25 μm | X |
| 1 | 267 | 7 | 267 | 2 | 605 | 12 | 267 | 7 |
| 2 | 240 | 18 | 240 | 35 | 395 | 5 | 240 | 18 |
| 3 | 342 | 11 | 342 | 8 | 374 | 7 | 342 | 11 |

### 3.3 Conclusion of the Third Round of Formula Screening

There are no significant differences in the appearance, concentration, purity, activity and sub-visible particles of the samples, and the formulas show good stability. Tween can promote the solubility, and for a high-concentration formulation, the increase of the concentration of Tween can effectively mitigate the increase of the number of sub-visible particles, and the 0.04% o formula is finally selected.

### Example 4. Influencing Factor Study

### 4.1 Procedures

This example was performed with the antibody toripalimab. The sample was concentrated by ultrafiltration using a Millipore Pellicon 21 membrane (0.11 m²) to a concentration of about 180 mg/mL, and dialyzed against the corresponding formula 2 shown in Table 3 to a final concentration of about 180 mg/mL, followed by the addition of polysorbate 80 (11) at the corresponding concentration. The solution was aseptically filled into 1 mL pre-filled syringes at 1.1 mL/syringe on a super-clean bench, and tested for the influencing factor. The specific scheme is shown in Table 30.

TABLE 30
Experimental scheme for influencing factors

| Conditions | T0 | Sampling time points | | |
|---|---|---|---|---|
| Horizontal shaking (room | X, Y | Day 5 | Day 10 | |
| temperature, 60-100 rpm) | | X, Y | X, Y | |
| Repeated freezing/thawing | | 3 C. | 5 C. | |
| (room temperature/−40° C.) | | X, Y | X, Y | |
| Illumination (25° C., | | Day 5 | Day 10 | Day 10 (dark) |
| 4500 ± 500 lux) | | X, Y | X, Y | X, Y |

Note:
X indicates: appearance, protein content, SEC-HPLC, R-CE-SDS, NR-CE-SDS, and CEX-HPLC
Y indicates: cell activity, binding activity, and HIAC

### 4.2 Results

### 4.2.1 Summary of Results of Horizontal Shaking Test

According to the results in Table 31, all the samples showed no significant change in protein concentration, appearance, purity by SEC-HPLC, purity by R-CE-SDS, purity by NR-CE-SDS, purity by CEX-HPLC, binding activity and cell activity.

TABLE 31
Summary of results of horizontal shaking test

| Test item | | T0 | Day 5 | Day 10 |
|---|---|---|---|---|
| Appearance | | CL, SO, FVP | CL, SO, FVP | CL, SO, FVP |
| Concentration (mg/mL) | | 170.7 | 171.1 | 169.1 |
| Purity by | Polymer % | 0.3 | 0.3 | 0.3 |
| SEC-HPLC | Monomer % | 99.7 | 99.7 | 99.7 |
| | Fragment % | 0.0 | 0.1 | 0.1 |
| Purity by R-CE-SDS | | 97.3 | 97.2 | 97.2 |
| Purity by NR-CE-SDS | | 99.5 | 99.5 | 99.5 |
| Purity by | Acid peak % | 13.4 | 13.7 | 13.9 |
| CEX-HPLC | Main peak % | 80.4 | 80.3 | 80.3 |
| | Basic peak % | 6.2 | 6.1 | 5.8 |
| Binding activity (%) | | 88 | 92 | 99 |
| Cell activity (%) | | 88.2 | 95.4 | 96.0 |
| HIAC | >10 μm | 25 | 42 | 20 |
| | >25 μm | 4 | 4 | 4 |

Note:
“CL” indicates colorless,
“SO” indicates slight opalescence, and
“FVP” indicates free of visible particles

### 4.2.2 Results of Freezing/Thawing Test

According to the results in Table 32, all the samples showed no significant change in protein concentration, appearance, purity by SEC-HPLC, purity by R-CE-SDS, purity by NR-CE-SDS, purity by CEX-HPLC, binding activity and cell activity.

TABLE 32
Summary of results of freezing/thawing test

| Test item | | T0 | 3 cycles | 5 cycles |
|---|---|---|---|---|
| Appearance | | CL, SO, FVP | CL, SO, FVP | CL, SO, FVP |
| Concentration (mg/mL) | | 176.0 | 173.1 | 169.1 |
| Purity by | Polymer % | 0.4 | 0.2 | 0.2 |
| SEC-HPLC | Monomer % | 99.6 | 99.8 | 99.8 |
| | Fragment % | 0.0 | 0.0 | 0.0 |
| Purity by R-CE-SDS | | 99.2 | 99.5 | 99.4 |
| Purity by NR-CE-SDS | | 97.0 | 96.9 | 96.8 |
| Purity by | Acid peak % | 15.0 | 15.3 | 15.6 |
| CEX-HPLC | Main peak % | 78.4 | 77.6 | 77.5 |
| | Basic peak % | 6.6 | 6.9 | 6.9 |
| Binding activity (%) | | 116 | 99 | 111 |
| Cell activity (%) | | 103 | 104 | 120 |
| HIAC | >10 μm | 240 | 249 | 250 |
| | >25 μm | 18 | 18 | 4 |

Note:
“CL” indicates colorless,
“SO” indicates slight opalescence, and
“FVP” indicates free of visible particles

### 4.2.3 Results of Illumination Test

According to the results in Table 33, all the samples showed no significant change in protein concentration, appearance, purity by SEC-HPLC, purity by R-CE-SDS, purity by NR-CE-SDS, purity by CEX-HPLC, binding activity and cell activity.

TABLE 33
Summary of results of illumination test

| Test item | | T0 | Day 5 | Day 10 | Day 10 (dark) |
|---|---|---|---|---|---|
| Appearance | | CL, SO, FVP | CL, SO, FVP | CL, SO, FVP | CL, SO, FVP |
| Concentration (mg/mL) | | 174.8 | 173.4 | 176.1 | 171.8 |
| Purity by | Polymer % | 0.3 | 0.4 | 0.5 | 0.4 |
| SEC-HPLC | Monomer % | 99.7 | 99.5 | 99.5 | 99.6 |
| | Fragment % | 0.0 | 0.0 | 0.0 | 0.0 |
| Purity by R-CE-SDS | | 97.2 | 97.0 | 96.8 | 97.0 |
| Purity by NR-CE-SDS | | 99.5 | 99.5 | 99.5 | 99.6 |
| Purity by | Acid peak % | 15.4 | 17.5 | 18.2 | 16.4 |
| CEX-HPLC | Main peak % | 78.3 | 73.7 | 72.9 | 77.5 |
| | Basic peak % | 6.3 | 8.8 | 8.9 | 6.1 |
| Binding activity (%) | | 99 | 103 | 102 | 100 |
| Cell activity (%) | | 92.9 | 90.3 | 87.8 | 91.2 |
| HIAC | >10 μm | 30 | 14 | 70 | 167 |
| | >25 μm | 5 | 0 | 2 | 5 |

Note:
“CL” indicates colorless,
“SO” indicates slight opalescence, and
“FVP” indicates free of visible particles

In conclusion, by investigating different buffer systems, different pH conditions, different antibody concentrations and different excipient compositions, the target pH range is controlled to be 5.9-6.1, the osmotic pressure range is controlled to be 260-320 mOsm/kg, and the optimal formula of the formulation is determined as follows: 20 mM histidine buffer (pH 6.0), 150 mM arginine hydrochloride and 0.04% o polysorbate 80 (11).

### Example 5. Long-Term Stability Study

Liquid pharmaceutical products including therapeutic antibodies usually need to be stored at 2-8° C., so it is important for the formulation to maintain high stability during the long-term storage. According to the above screening results, the formula of the formulation is as follows: about 180 mg/mL of antibody toripalimab, 20 mM histidine buffer (pH 6.0), 150 mM arginine hydrochloride and 0.04% o polysorbate 80 (II). This formula was used for subsequent production and long-term stability investigation.

Two batches of finished products were selected and stored for 6 months at 2-8° C., and then the samples were analyzed. Stability was evaluated by the following parameters: (a) appearance; (b) pH; (c) molecular weight of the antibody by CE-SDS (capillary electrophoresis-sodium dodecyl sulfate) method; (d) content of antibody monomers or polymers by SEC-HPLC; (e) mainly charged form, acidic forms, or basic forms of the antibody by CEX-HPLC; (f) binding activity of the antibody by an ELISA method; and (g) protein content.

The results showed that the two batches of finished products showed no significant change in appearance, pH, protein content, purity (size-exclusion high-performance liquid chromatography, SEC-HPLC), CEX-HPLC (cation exchange high-performance liquid chromatography), R-CE-SDS (reduced electrophoresis), NR-CE-SDS (non-reduced electrophoresis) and biological activity. The specific results are shown in Table 34. The results suggest that the two batches of finished products have very good stability during storage for 0-6 months at 2-8° C.

TABLE 34
Long-term stability data of formulations

| | Time | Result | |
|---|---|---|---|
| Test item | (month) | Batch 1 | Batch 2 |
| Appearance | 0 | Colorless and clear liquid | Colorless and clear |
| | | with slight opalescence | liquid |
| | 3 | Colorless and clear liquid | Colorless liquid with |
| | | with slight opalescence | slight opalescence |
| | 6 | Colorless and clear liquid | Colorless liquid and |
| | | with slight opalescence | almost clear |
| pH | 0 | 6.1 | 6.0 |
| | 3 | 6.1 | 6.1 |
| | 6 | 6.0 | 6.1 |
| SEC-HPLC monomer | 0 | 99.5 | 99.5 |
| content (%) | 3 | 99.4 | 99.4 |
| | 6 | 99.4 | 99.4 |
| SEC-HPLC polymer | 0 | 0.5 | 0.5 |
| content (%) | 3 | 0.6 | 0.6 |
| | 6 | 0.6 | 0.6 |
| CEX-HPLC main peak | 0 | 79.4 | 81.5 |
| content (%) | 3 | 79.3 | 80.8 |
| Appearance | 0 | Colorless and clear liquid | Colorless and clear |
| | | with slight opalescence | liquid |
| | 3 | Colorless and clear liquid | Colorless liquid with |
| | | with slight opalescence | slight opalescence |
| | 6 | Colorless and clear liquid | Colorless liquid and |
| | | with slight opalescence | almost clear |
| pH | 0 | 6.1 | 6.0 |
| | 3 | 6.1 | 6.1 |
| | 6 | 6.0 | 6.1 |
| | 6 | 80.0 | 80.2 |
| CEX-HPLC acidic peak | 0 | 12.9 | 9.5 |
| content (%) | 3 | 13.0 | 10.9 |
| | 6 | 12.7 | 12.3 |
| CEX-HPLC basic peak | 0 | 7.8 | 9.0 |
| content (%) | 3 | 7.7 | 8.2 |
| | 6 | 7.3 | 7.5 |
| R-CE-SDS content | 0 | 99.4 | 99.4 |
| (%) | 3 | 99.5 | 99.3 |
| | 6 | 99.6 | 99.5 |
| NR-CE-SDS content | 0 | 97.9 | 97.1 |
| (%) | 3 | 97.2 | 96.4 |
| | 6 | 96.2 | 96.6 |
| Biological activity | 0 | 86 | 99 |
| (by ELISA, %) | 3 | 102 | 107 |
| | 6 | 97 | 96 |
| Protein content | 0 | 185.9 | 181.0 |
| (by ultraviolet | 3 | 184.2 | 183.7 |
| spectrophotometry) | 6 | 184.9 | 186.6 |

### Example 6. Accelerated Stability Study of Formulations

Two batches of finished products were selected and stored for 0-6 months under the conditions of 25±2° C. and 60%±5% relative humidity (RH), and then the samples were analyzed. The formula of the formulation is as follows: about 180 mg/mL of antibody toripalimab, 20 mM histidine buffer (pH 6.0), 150 mM arginine hydrochloride and 0.04% polysorbate 80 (II).

As shown in Table 35, the finished product has higher stability against protein degradation, and the resulting degradation kinetic parameters measured at 25±2° C. meet the requirements for storage at room temperature for up to 6 months.

TABLE 35
Accelerated stability data of formulations

| | Time | Result | |
|---|---|---|---|
| Test item | (month) | Batch 1 | Batch 2 |
| Appearance | 0 | Colorless and clear liquid | Colorless and clear |
| | | with slight opalescence | liquid |
| | 3 | Colorless liquid with | Colorless liquid with |
| | | slight opalescence | slight opalescence |
| | 6 | Colorless liquid with | Colorless liquid and |
| | | slight opalescence | almost clear |
| pH | 0 | 6.1 | 6.0 |
| | 3 | 6.1 | 6.1 |
| | 6 | 6.1 | 6.2 |
| SEC-HPLC monomer | 0 | 99.5 | 99.5 |
| content (%) | 3 | 99.1 | 99.2 |
| | 6 | 99.1 | 99.1 |
| SEC-HPLC polymer | 0 | 0.5 | 0.5 |
| content (%) | 3 | 0.8 | 0.7 |
| | 6 | 0.8 | 0.9 |
| CEX-HPLC main peak | 0 | 79.4 | 81.5 |
| content (%) | 3 | 76.4 | 77.9 |
| | 6 | 72.1 | 72.0 |
| CEX-HPLC acidic peak | 0 | 12.9 | 9.5 |
| content (%) | 3 | 18.5 | 16.9 |
| Appearance | 0 | Colorless and clear liquid | Colorless and clear |
| | | with slight opalescence | liquid |
| | 3 | Colorless liquid with | Colorless liquid with |
| | | slight opalescence | slight opalescence |
| | 6 | Colorless liquid with | Colorless liquid and |
| | | slight opalescence | almost clear |
| pH | 0 | 6.1 | 6.0 |
| | 3 | 6.1 | 6.1 |
| | 6 | 6.1 | 6.2 |
| | 6 | 24.4 | 24.1 |
| CEX-HPLC basic peak | 0 | 7.8 | 9.0 |
| content (%) | 3 | 5.1 | 5.2 |
| | 6 | 3.4 | 3.9 |
| R-CE-SDS content | 0 | 99.4 | 99.4 |
| (%) | 3 | 98.9 | 99.2 |
| | 6 | 98.6 | 98.6 |
| NR-CE-SDS content | 0 | 97.9 | 97.1 |
| (%) | 3 | 96.6 | 96.0 |
| | 6 | 95.2 | 95.6 |
| Biological activity | 0 | 86 | 99 |
| (by ELISA, %) | 3 | 108 | 111 |
| | 6 | 103 | 106 |
| Protein content | 0 | 185.9 | 181.0 |
| (by ultraviolet | 3 | 187.2 | 181.7 |
| spectrophotometry) | 6 | 186.3 | 184.3 |
| Insoluble microparticles | 0 | ≥10 μm: 0 microparticles/ | ≥10 μm: 46 microparticles/ |
| | | vial; ≥25 μm: 0 | vial; ≥25 μm: 0 |
| | | microparticles/vial | microparticles/vial |
| Appearance | 0 | Colorless and clear liquid | Colorless and clear |
| | | with slight opalescence | liquid |
| | 3 | Colorless liquid with | Colorless liquid with |
| | | slight opalescence | slight opalescence |
| | 6 | Colorless liquid with | Colorless liquid and |
| | | slight opalescence | almost clear |
| pH | 0 | 6.1 | 6.0 |
| | 3 | 6.1 | 6.1 |
| | 6 | 6.1 | 6.2 |
| | 6 | ≥10 μm: 2 microparticles/ | ≥10 μm: 6 microparticles/ |
| | | vial; ≥25 μm: 0 | vial; ≥25 μm: 2 |
| | | microparticles/vial | microparticles/vial |

### Example 7. Comparison of Pharmacokinetics of Subcutaneous and Intravenous Formulations in Cynomolgus Monkeys

### 7.1 Objective

The cynomolgus monkeys were subcutaneously injected with JS001 (toripalimab) subcutaneous formulation at different doses, and detected for the drug concentration in serum, to evaluate the primary pharmacokinetic characteristics of the JS001 subcutaneous formulation (with a formula of FS2-2 in Example 2); meanwhile, an intravenous administration group was set, and the formula of the JS001 intravenous formulation was as follows: about 40 mg/mL JS001, about 20 mM citrate buffer (about pH 6.0), about 150 mM mannitol, about 50 mM sodium chloride, and about 0.02% o polysorbate 80, to evaluate the pharmacokinetic characteristics of the JS001 through different routes of administration, and preliminarily investigate the bioavailability.

### 7.2 Preparation of Test Sample

The amount of the test sample required was calculated according to the recent body weight of the cynomolgus monkey, the dose administered and the drug content. The intravenous formulation was prepared by diluting the test sample to 0.8 mg/mL with 0.9% sodium chloride injection under sterile conditions. In this experiment, the prepared intravenous formulation was required to be immediately administered to the test animals by intravenous infusion for 30 min, with a constant-speed infusion pump recommended. The subcutaneous formulation was prepared by diluting the test sample to the required concentration with placebo (self-made by Junmeng), and administered at a concentration of 0.5 mL/kg and injected on the lateral thigh of the hind limb.

### 7.3 Animal Selection, Dosing Design and Grouping

In this experiment, the cynomolgus monkeys weighing 2.5-5 kg were selected and randomly grouped with 3 cynomolgus monkeys in each group. The grouping and administration regimen of the cynomolgus monkey are shown in Table 36. The group B was administered once with the JS001 subcutaneous formulation at a dose of 4 mg/kg; and the group A was administered with the JS001 intravenous formulation at a dose of 4 mg/kg.

TABLE 36
Grouping and administration regimen of the cynomolgus monkey

| | | | | | Infusion | | | |
|---|---|---|---|---|---|---|---|---|
| | Test | Administration | Administration | | rate per | | | |
| | sample | Dose | concentration | Route of | unit time | Time of | Animal No. | |
| Groups | batch No. | (mg/kg) | (mg/mL) | administration | (μL/kg/min) | infusion | Female | Male |
| A | JS001 | 4 | 0.8 | iv | 166.67 | 30 min | AF01 | AM01/AM02 |
| | intravenous | | | | | | | |
| | formulation | | | | | | | |
| B | JS001 | 4 | 8 | sc | NA | NA | CF01 | CM01/CM02 |
| | subcutaneous | | | | | | | |
| | formulation | | | | | | | |

Note:
sc: subcutaneous injection; 2. iv: intravenous injection; 3: NA: none.

### 7.4 Blood Sampling and Results

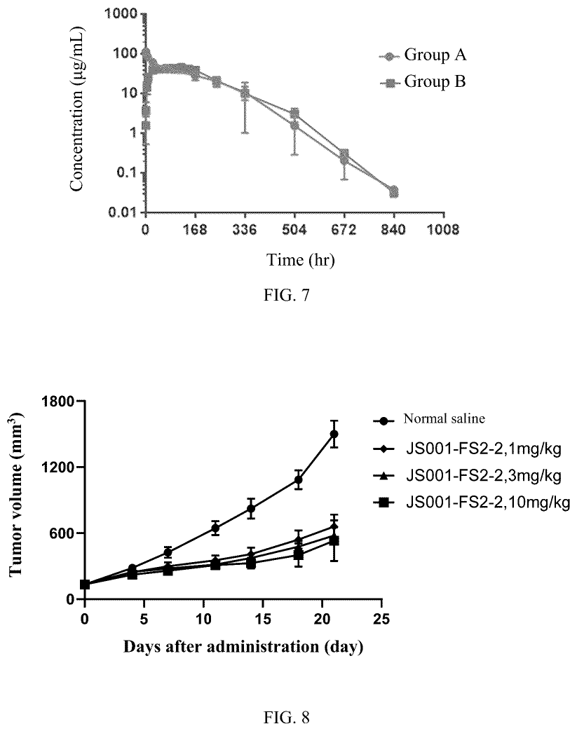

Whole blood samples (about 1 mL of blood collected for PK) were taken from the vein of non-administered limbs of cynomolgus monkeys and put into marked sample tubes, which were put into an ice box. After the blood was naturally coagulated, the tubes were centrifuged at 1500×g for 10 min at 2-8° C. in a centrifuge, and the serum were isolated and added into EP tubes marked with sample number. PK samples were each aliquoted into 2 tubes, one for testing and the other one for back-up. After processing, the samples were stored in a refrigerator at −60 to −90° C. After the sampling, the samples were transported to a biological sample analysis department by adopting cold chain logistics, accompanied with relevant transportation records and temperature control records to ensure that the samples were kept unthawed during the transportation. The pharmacological parameters were calculated using a non-compartmental model of WinNonlin v 6.4 (Pharsight Inc.) software. AUC₍₀₋ₜ₎ was calculated by Linear Trapezoidal Linear Interpolation. The parameters such as elimination rate constant Kₑₗ, half-life t_{1/2}, time to peak Tₘₐₓ, peak concentration Cₘₐₓ, drug exposure AUC₍₀₋ₜ₎, apparent volume of distribution V_{d}, Systemic clearance CLs, and mean residence time MRT were reported. The mean drug concentrations in serum in the two groups are shown in Table 37, the mean plasma concentration-time curves of the two groups are shown in FIG. 7, and the mean pharmacokinetic results in serum of the two groups are shown in Table 38.

TABLE 37
Mean drug concentrations in serum in the groups A and B

| | Group A | Group B |
|---|---|---|
| Time | Mean ± standard deviation | Mean ± standard deviation |
| (hr) | (μg/mL) | (μg/mL) |
| 0 | BQL | BQL |
| 0.5 | 116 ± 8.08 | 1.60 ± 1.06 |
| 1 | NA | 3.81 ± 2.36 |
| 2 | 96.6 ± 6.72 | NA |
| 4 | NA | 14.6 ± 4.95 |
| 6 | 85.4 ± 15.1 | NA |
| 8 | NA | 26.5 ± 6.58 |
| 24 | 62.4 ± 9.29 | 39.2 ± 7.53 |
| 48 | NA | 41.8 ± 7.33 |
| 72 | 41.8 ± 4.86 | 43.7 ± 8.14 |
| 96 | NA | 43.9 ± 9.83 |
| 120 | NA | 45.1 ± 11.8 |
| 144 | NA | 42.6 ± 8.71 |
| 168 | 28.7 ± 7.09 | 39.8 ± 9.80 |
| 240 | 20.8 ± 5.93 | 21.7 ± 5.56 |
| 336 | 11.1 ± 4.20 | 10.3 ± 9.24 |
| 504 | 1.58 ± 1.28 | 3.13 ± 1.20 |
| 672 | 0.209 ± 0.139 | 0.319 ± 0.0474 |
| 840 | 0.0391 | 0.0330 ± 0.00714 |
| 1008 | BQL | BQL |

Note:
NA indicates not detected and hr indicates hour.

TABLE 38
Pharmacokinetic parameters of cynomolgus
monkey (mean ± standard deviation)

| Parameter | Unit | Group A | Group B |
|---|---|---|---|
| Kₑₗ | 1/hr | 0.0141 ± 0.00595 | 0.0135 ± 0.00191 |
| t_{1/2} | hr | 54.9 ± 21.2 | 52.1 ± 7.21 |
| Tₘₐₓ | hr | 0.500 | 48.0~120 |
| Cₘₐₓ | μg/mL | 116 ± 8.08^{aaa} | 48.3 ± 7.32 |
| AUC₍₀₋ₜ₎ | hr*μg/mL | 12300 ± 1910 | 11800 ± 700 |
| AUC_{inf} | hr*μg/mL | 12300 ± 1910 | 12000 ± 919 |
| AUCₑₓₜᵣ | % | 0.0591 ± 0.0344 | 0.0209 ± 0.00573 |
| V_{d} | mL/kg | 26.6 ± 11.3 | 25.2 ± 1.48 |
| CLₛ | mL/hr/kg | 0.331 ± 0.0534 | 0.336 ± 0.0269 |
| MRT | hr | 141 ± 8.33^{aa} | 185 ± 7.07 |
| F | % | NA | 95.9 |

Note:
NA indicates none,
hr indicates hour;
^{aa}indicates P < 0.01, and
^{aaa}indicates P < 0.001, group A vs group B.

The results showed that when the cynomolgus monkeys were subcutaneously injected once with test drug JS001 at a dose of 4 mg/kg, the AUC₍₀₋ₜ₎ was 11800±700 hr×μg/mL; when the cynomolgus monkeys were intravenously injected once with the test drug JS001 at a dose of 4 mg/kg, the AUC₍₀₋ₜ₎ was 12300±1910 hr×μg/mL; the bioavailability of JS001 injected subcutaneously was 95.9%; the trends of change in the mean plasma concentration-time curves of the two groups for subcutaneous and intravenous administrations of the test drug JS001, respectively, were substantially consistent. Therefore, the JS001 intravenous formulation has equivalent pharmacokinetics to the JS001 subcutaneous injection formulation, and the JS001 subcutaneous injection formulation can improve the compliance and administration convenience for tumor patients.

### Example 8. Inhibitory Effect of Subcutaneous Injection Formulation on Growth of Transplanted MC38 Tumor in hPD-1 Humanized Mice

### 8.1 Objective

To evaluate the anti-tumor effect of JS001 (toripalimab) subcutaneous injection formulation (with a formula of FS2-2 in Example 2) of the present disclosure on the mouse colon cancer MC38 subcutaneous xenograft model.

### 8.2 Procedures

Female hPD-1 humanized mice aged 6-7 weeks (Biocytogen Jiangsu Co., Ltd.) were subcutaneously inoculated with 1×10⁶ MC38 cells (0.1 mL/mouse) on the right dorsal side. When the mean tumor volume was about 134 mm³, 24 animals were selected and divided randomly into 4 groups based on tumor volume, with 6 animals in each group. The groups were as follows: a negative control group in which normal saline was administered and JS001-FS2-2 treatment groups in which FS2-2 formulations of 1 mg/kg, 3 mg/kg, and 10 mg/kg were administered, respectively.

The administration was performed on the day of grouping, and all groups were subjected to administration by subcutaneous injection at the neck, twice a week for consecutive 6 times. The experiment ended 3 days after the last administration. Tumor volume and body weight of mice were measured and recorded twice a week. At the end of the experiment, mice were euthanized and tumor inhibition TGI % (TGI %=[1−(Ti−T0)/(Vi−V0)]×100%) was calculated. (Ti: mean tumor volume of the treatment group on day i of administration, T0: mean tumor volume of the treatment group on day 0 of administration; Vi: mean tumor volume of the negative control group on day i of administration, V0: mean tumor volume of the negative control group on day 0 of administration).

The results are shown in FIG. 8. The results showed that the mean tumor volume of the negative control group was 1501±122 mm³ on day 21 after the start of the administration. The values of the mean tumor volume for JS001-FS2-2 formulations at doses of 1 mg/kg, 3 mg/kg and 10 mg/kg were 661±108 mm³, 578±75 mm³, 531±184 mm³, respectively, with TGI % of 61.5%, 67.5% and 71.0%, respectively. It suggests that JS001-FS2-2 formulations at doses of 1 mg/kg, 3 mg/kg and 10 mg/kg significantly inhibit the increase of volume of the transplanted MC38 tumor in hPD-1 humanized mice, and exhibit a good dose-response relationship.

1. A pharmaceutical composition, comprising:
(1) a buffer; and
(2) an anti-PD-1 antibody or an antigen-binding fragment thereof;
wherein the anti-PD-1 antibody or the antigen-binding fragment thereof comprises an LCDR1, an LCDR2 and an LCDR3 having amino acid sequences set forth in SEQ ID NO: 1, SEQ ID NO: 2 and SEQ ID NO: 3, respectively, and an HCDR1, an HCDR2 and an HCDR3 having amino acid sequences set forth in SEQ ID NO: 4, SEQ ID NO: 5 and SEQ ID NO: 6, respectively, wherein
the anti-PD-1 antibody or the antigen-binding fragment thereof has a concentration of about 100-250 mg/mL.

2. The pharmaceutical composition according to claim 1, wherein the buffer is selected from one or more of an acetic acid buffer, a citric acid buffer and a histidine buffer; wherein the buffer is a histidine buffer selected from a histidine-histidine hydrochloride buffer and a histidine-histidine acetate buffer; wherein the buffer has a concentration of about 10-50 mM; and wherein the buffer has a pH of about 5.0-6.5.

3. The pharmaceutical composition according to claim 1, wherein the pharmaceutical composition further comprises a stabilizer selected from one or more of arginine, an arginine salt, sodium chloride, mannitol, sorbitol, sucrose, glycine and trehalose; wherein the arginine salt is arginine hydrochloride; and wherein the stabilizer has a concentration of about 100-250 mM.

4. The pharmaceutical composition according to claim 3, wherein the stabilizer is arginine or an arginine salt having a concentration of about 120-220 mM; or the stabilizer is a combination of arginine hydrochloride having a concentration of about 30-100 mM and sucrose having a concentration of about 100-180 mM; or the stabilizer is a combination of arginine hydrochloride having a concentration of about 30-100 mM and glycine having a concentration of about 50-150 mM.

5. The pharmaceutical composition according to claim 1, wherein the pharmaceutical composition further comprises a surfactant selected from one or more of polysorbate 80, polysorbate 20 and poloxamer 188; wherein based on w/v, the surfactant has a concentration of about 0.01%-0.1%.

6. The pharmaceutical composition according to claim 1, wherein the anti-PD-1 antibody or the antigen-binding fragment thereof comprises a light chain variable region set forth in SEQ ID NO: 7 and a heavy chain variable region set forth in SEQ ID NO: 8.

7. The pharmaceutical composition according to claim 1, comprising components as shown in any one of (1) to (8) below:
(1) (a) about 150-250 mg/mL of the anti-PD-1 antibody or the antigen-binding fragment thereof, (b) about 10-30 mM histidine buffer having a pH of about 5.5-6.5; (c) about 120-220 mM arginine or arginine salt; and (d) about 0.01%-0.1% polysorbate 80; or
(2) (a) about 150-250 mg/mL of the anti-PD-1 antibody or the antigen-binding fragment thereof; (b) about 10-30 mM histidine buffer having a pH of about 5.5-6.5; (c) a stabilizer, being a combination of arginine hydrochloride having a concentration of about 30-100 mM and sucrose having a concentration of about 100-180 mM; and (d) about 0.01%-0.1% polysorbate 80; or
(3) (a) about 150-250 mg/mL of the anti-PD-1 antibody or the antigen-binding fragment thereof; (b) about 10-30 mM histidine buffer having a pH of about 5.5-6.5; (c) a stabilizer, being a combination of arginine hydrochloride having a concentration of about 30-100 mM and glycine having a concentration of about 50-150 mM; and (d) about 0.01%-0.1% polysorbate 80; or
(4) (a) about 150-200 mg/mL of the anti-PD-1 antibody or the antigen-binding fragment thereof; (b) about 10-30 mM acetic acid buffer having a pH of about 5.5-6.0; (c) about 130-180 mM arginine or arginine hydrochloride; and (d) about 0.02%-0.08% polysorbate 80; or
(5) (a) about 150-200 mg/mL of the anti-PD-1 antibody or the antigen-binding fragment thereof, (b) about 10-30 mM acetic acid buffer having a pH of about 5.5-6.0; (c) a stabilizer, being a combination of arginine hydrochloride having a concentration of about 30-70 mM and sucrose having a concentration of about 110-170 mM; and (d) about 0.02%-0.08% polysorbate 80; or
(6) (a) about 150-200 mg/mL of the anti-PD-1 antibody or the antigen-binding fragment thereof; (b) about 10-30 mM acetic acid buffer having a pH about 5.5-6.0; (c) a stabilizer, being a combination of arginine hydrochloride having a concentration of about 30-70 mM and glycine having a concentration of about 80-120 mM; and (d) about 0.02%-0.08% polysorbate 80; or
(7) (a) about 180 mg/mL of an anti-PD-1 antibody comprising a light chain amino acid sequence set forth in SEQ ID NO: 9 and a heavy chain amino acid sequence set forth in SEQ ID NO: 10; (b) about 20 mM histidine buffer having a pH of about 6.0; (c) about 140 mM arginine hydrochloride; and (d) about 0.02% polysorbate 80; or
(8) (a) about 180 mg/mL of an anti-PD-1 antibody comprising a light chain amino acid sequence set forth in SEQ ID NO: 9 and a heavy chain amino acid sequence set forth in SEQ ID NO: 10; (b) about 20 mM histidine buffer having a pH of about 6.0; (c) about 150 mM arginine hydrochloride; and (d) about 0.04% polysorbate 80.

8. An injection comprising the pharmaceutical composition according to claim 1 and a sodium chloride solution or a glucose solution; wherein the sodium chloride solution has a concentration of about 0.85%-0.9% (w/v); the glucose solution has a concentration of about 5%-25% (w/v); wherein in the injection, the anti-PD-1 antibody has a concentration of about 0.5-50 mg/mL; and wherein, the injection has a pH of about 5.0-6.5.

9. The pharmaceutical composition according to claim 1, wherein the pharmaceutical composition is administered by subcutaneous injection.

10. A method for treating a disease or a disorder by eliminating, inhibiting or reducing PD-1 activity, which comprises administering to a subject in need thereof the pharmaceutical composition according to claim 1, wherein the disease or disorder is selected from cancer, infectious diseases and inflammatory diseases.

11. A method for treating a disease or a disorder by eliminating, inhibiting or reducing PD-1 activity, which comprises administering to a subject in need thereof the injection according to claim 8; wherein the disease or disorder is selected from cancer, infectious diseases and inflammatory diseases.

12. The pharmaceutical composition according to claim 1, wherein the pharmaceutical composition has a pH of about 5.0-6.5.

13. The pharmaceutical composition according to claim 1, wherein the pharmaceutical composition has an osmotic pressure in a range of 260-320 mOsm/kg.

14. The pharmaceutical composition according to claim 2, wherein the buffer is a histidine buffer selected from a histidine-histidine hydrochloride buffer and a histidine-histidine acetate buffer.

15. The pharmaceutical composition according to claim 4, wherein the stabilizer is arginine or an arginine salt having a concentration of about 130-180 mM; or the stabilizer is a combination of arginine hydrochloride having a concentration of about 30-70 mM and sucrose having a concentration of about 110-170 mM; or the stabilizer is a combination of arginine hydrochloride having a concentration of about 30-70 mM and glycine having a concentration of about 80-120 mM.

16. The pharmaceutical composition according to claim 15, wherein the arginine salt is arginine hydrochloride.

17. The pharmaceutical composition according to claim 5, wherein the surfactant has a concentration of about 0.02%-0.08%.

18. The pharmaceutical composition according to claim 6, wherein the anti-PD-1 antibody comprises a light chain amino acid sequence set forth in SEQ ID NO: 9 and a heavy chain amino acid sequence set forth in SEQ ID NO: 10.

19. The injection according to claim 8, wherein the anti-PD-1 antibody has a concentration of about 0.5-20 mg/mL; and wherein the injection has a pH of about 5.5-6.2.

20. The injection according to claim 8, wherein the injection is administered by subcutaneous injection.


SEQUENCE LISTING (10 sequences)

SEQ ID NO: 1 - protein, 16 aa, synthetic construct
RSSQSIVHSNGNTYLE

SEQ ID NO: 2 - protein, 7 aa, synthetic construct
KVSNRFS

SEQ ID NO: 3 - protein, 9 aa, synthetic construct
FQGSHVPLT

SEQ ID NO: 4 - protein, 5 aa, synthetic construct
DYEMH

SEQ ID NO: 5 - protein, 17 aa, synthetic construct
VIESETGGTAYNQKFKG

SEQ ID NO: 6 - protein, 16 aa, synthetic construct
EGITTVATTYYWYFDV

SEQ ID NO: 7 - protein, 112 aa, synthetic construct [sequence omitted]

SEQ ID NO: 8 - protein, 125 aa, synthetic construct [sequence omitted]

SEQ ID NO: 9 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK